(19) United States
(12) Patent Application Publication
(10) Pub. No.: US 2026/0355361 A1
(43) Pub. Date: Nov. 12, 2026

(54) METHODS FOR DIAGNOSIS AND TREATMENT OF PULMONARY HYPERTENSION

(71) Applicant: THE METHODIST HOSPITAL, Houston, TX (US)

(72) Inventors: Zheng Li, Bellaire, TX (US); Feng Li, Sugar Land, TX (US); Ashrith Guha, Houston, TX (US)

(21) Appl. No.: 18/998,958

(22) PCT Filed: Jul. 27, 2023
(86) PCT No.: PCT/US2023/028790

§ 371 Date: Jan. 27, 2025

Related U.S. Application Data
(60) Provisional application No. 63/392,578, filed on Jul. 27, 2022.

(51) Int. Cl. A61K 51/04; A61K 45/06; C07B 59/00; C07F 5/00; C07F 5/06

(52) U.S. Cl. CPC A61K 51/0482; A61K 45/06; A61K 51/0455; C07B 59/004; C07F 5/003; C07F 5/069; A61K 2121/00; A61K 2123/00; C07B 2200/05

(57) ABSTRACT

A method is disclosed of detecting a disease associated with pulmonary vascular remodeling in a subject. The method can comprise administering to the subject a compound of Formula I or Formula II. The method can further comprise imaging the compound in lungs of the subject using positron emission tomography to determine uptake of the compound in pulmonary vasculature of the lungs, wherein retention of the compound in the pulmonary vasculature reflects vascular remodeling. Methods of treating a disease associated with pulmonary vascular remodeling or for imaging a pulmonary vasculature in a subject are also provided.

### CROSS-REFERENCE TO RELATED APPLICATIONS

This application claims the benefit of priority to U.S. Provisional Application No. 63/392,578, filed Jul. 27, 2022, the disclosure of which is incorporated herein by reference in its entirety.

### TECHNICAL FIELD

This disclosure relates to methods for the diagnosis and treatment of pulmonary hypertension and, more particularly, to the use of PET imaging agents that bind to vascular endothelial growth factor receptor 2 (VEGFR-2) in the diagnosis of pulmonary hypertension.

### BACKGROUND

Pulmonary hypertension (PH) is defined by increased pressure in the pulmonary vasculature that includes the pulmonary arterial and venous system. Pulmonary hypertension, when present with chronic heart and lung disease, increases the mortality of those patients by 4-5 fold.

One of the subtypes of PH, which involves only the arterial system (pulmonary arterial hypertension or PAH), is a deadly disease that, despite medical therapy, has a 5-year mortality rate of 50%. A significant reason for this high mortality is the poor accuracy of noninvasive echocardiography for diagnosis. Echocardiography is notoriously inaccurate, with a 45% false negative rate in population studies for the diagnosis of PH. There is exhaustion of 70% of the pulmonary vascular reserve by the time of diagnosis, leading to an irreversible trajectory of this disease. In addition, the diagnosis is based on a late manifestation of the disease (an increase in mean pulmonary artery pressure or mPAP) but not on the identification of the underlying pathophysiological process.

Thus, there is a clear need for an early diagnostic tool to accurately identify PH and monitor disease progression based on pathophysiological changes in the disease.

### SUMMARY

In accordance with the purposes of the disclosed subject matter, as embodied and broadly described herein, disclosed are methods of diagnosis and/or treatment of diseases associated with pulmonary vascular remodeling.

In one aspect, a method of detecting a disease associated with pulmonary vascular remodeling in a subject is provided, the method comprising:
(a) administering to the subject a compound of Formula I or Formula II

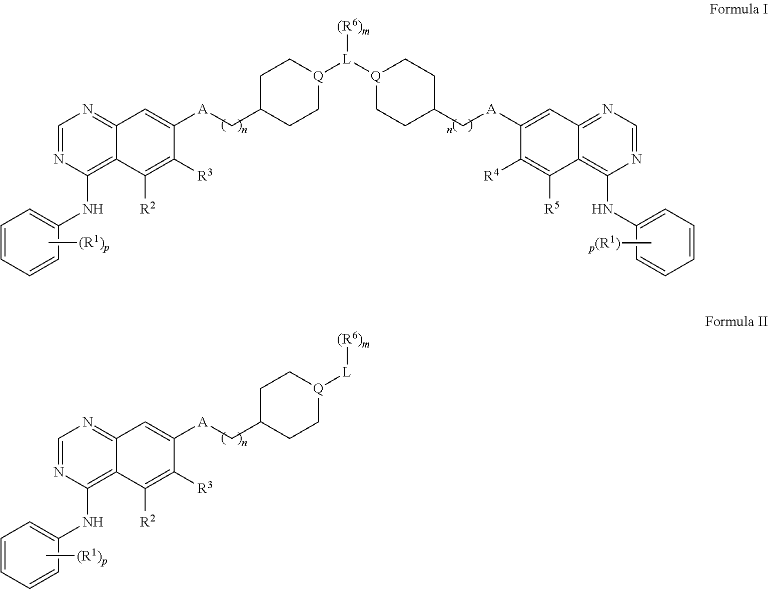

or a pharmaceutically acceptable salt thereof; and
(b) imaging the compound in the subject using positron emission tomography (PET) to detect uptake of the compound in a pulmonary vasculature of the subject;
wherein uptake of the compound in the pulmonary vasculature reflects vascular remodeling;
and wherein all variables are as defined herein.

In another aspect, a method of treating a disease associated with pulmonary vascular remodeling in a subject, the method comprising:
(a) administering to the subject a compound of Formula I or Formula II, or a pharmaceutically acceptable salt thereof;
(b) imaging the compound in the subject using positron emission tomography (PET) to detect uptake of the compound in a pulmonary vasculature of the subject; and
if retention of the compound is detected in the pulmonary vasculature in (b), then (c) administering to the subject a therapy for the disease associated with pulmonary vascular remodeling.

In another aspect, a compound is provided having the chemical formula

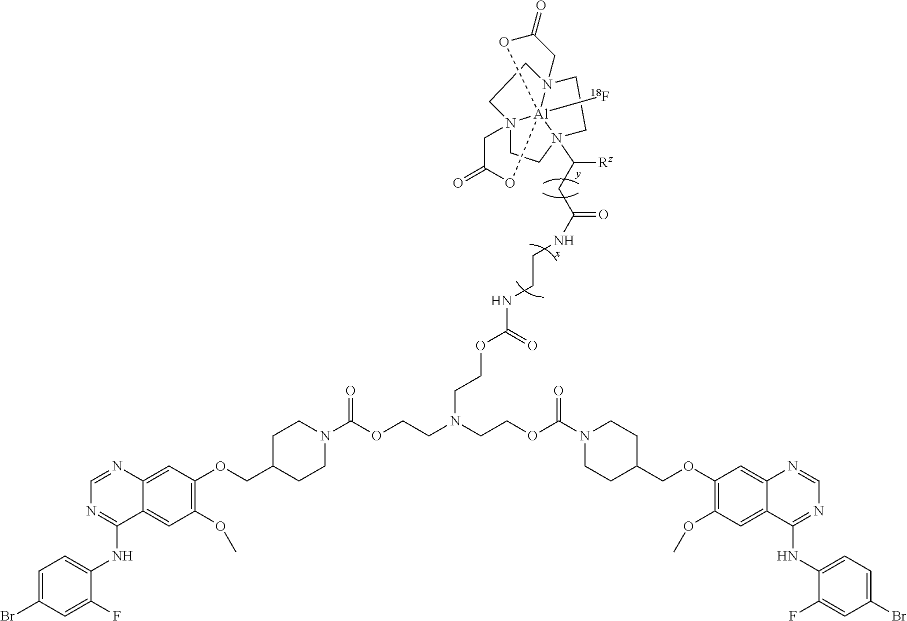

wherein all variables are as defined herein.
or a pharmaceutically acceptable salt thereof. Methods of diagnosing and/or treating diseases associated with pulmonary vascular remodeling using said compound are also provided.

Additional advantages will be set forth in part in the description which follows, and in part will be obvious from the description, or may be learned by practice of the subject matter disclosed herein. It is to be understood that both the foregoing general description and the following detailed description are exemplary and explanatory only and are not restrictive of the invention, as claimed.

### DESCRIPTION OF DRAWINGS

The accompanying figures, which are incorporated in and constitute a part of this specification, illustrate embodiments of the invention and together with the description, serve to explain the principles of the disclosed subject matter.

FIG. 1 shows the chemical structure of representative compounds as used in the disclosed methods.

FIGS. 2A-2C show the characterization of VEGFr2-specific binding (left). (FIG. 2A) Saturation curve of ⁶⁴Cu-vandetanib bound to U87 cells (K_{d}: 44.75±15.04 nM). (FIG. 2B) Saturation curve of ⁶⁴Cu-ZD-G2 bound to U87 cells. (K_{d}: 0.45±0.32 nM). (FIG. 2C) MicroPET/CT imaging of U87 tumor-bearing mice at 24 h post injection of 3.7 MBq of ⁶⁴Cu-vandetanib and ⁶⁴Cu-ZD-G2 (right). Tumors are indicated by arrowheads.

FIG. 3 shows staining of PAH human lung samples by VEGFr2 antibody and Cy5.5-ZD-G2.

FIGS. 4A-4B show optical imaging of PAH rats using Cy5.5-ZD-G2 probe. 10 nmol probe was injected into the rat prior to imaging. After 30 min of the injection, both optical imaging of the rat and major organs were collected, respectively (FIG. 4A). Afterwards, lung tissue was specifically collected and performed further histological study (FIG. 4B).

FIGS. 5A-5C shows PET imaging of PAH rats at 24 h post injection of ⁶⁴Cu-ZD-G2. (FIG. 5A) Development of PAH is induced by one-time administration of monocrotaline; ROI analysis of the rat lungs. (FIG. 5B) In the blocking study, co-injection of ⁶⁴Cu-ZD-G2 probe with excessive amount of VEGFr2 inhibitor effectively decreased the lung uptake in PAH mice, which demonstrated specific VEGFr2 targeting of the probe (FIG. 5C).

FIG. 6 shows radio-HPLC spectrum of ¹⁸F-ZD-G2 before and after incubation of mouse serum.

FIG. 7 shows representative PET images at 60 minutes after probe injection. Images of ¹⁸F-ZD-G2 probe and ¹⁸FDG were compared. Rats exposed to monocrotaline were used to develop pulmonary arterial hypertension rat model (MCT), while healthier rats were used as a control group.

FIG. 8 shows Masson trichrome stained histological sections of donor and Pulmonary Hypertension (PH) patient lung tissue. Nuclei appear in dark red, cytoplasm in light red and collagen in blue.

FIG. 9 shows Verhoeff von Gieson (VVG) stained histological sections of donor and Pulmonary Hypertension (PH) patient lung tissue. Wall thickness will be quantified by VVG staining.

FIG. 10 shows hematoxylin and eosin (HE) staining histological sections of donor and Pulmonary Hypertension (PH) patient lung tissue. Medial hypertrophy will be quantified by HE staining.

FIG. 11 shows VEGFR2 immunohistochemistry of sections of donor and Pulmonary Hypertension (PH) patient lung tissue. Brown color represent the VEGFR2 positive nuclei.

FIG. 12 shows H&E and VEGFR IHC staining of samples from different PAH groups. Different levels and distribution of VEGFR2 expression was shown in the PAH tissues.

FIG. 13 shows fluorescence staining of samples from different PAH groups using Cy5.5-ZD-G2.

FIG. 14 depicts a monocrotaline (MCT) rat model of pulmonary arterial hypertension (PAH).

FIGS. 15A and 15B depict Masson's trichrome stained lung sections of rats in the MCT rat model. A marked increase of collagen deposition was observed in lung sections of MCT week 3 and 4.

FIG. 16 depicts pulmonary arterial wall thickening by Verhof-van Giesen (VVG) staining of lunch sections of rats in the MCT rat model. Increased wall thickness is observed in MCT week 3 and 4.

FIG. 17 depicts right ventricular (RV) systolic pressure for rats in the MCT rat model.

FIG. 18 shows representative PET images at 60 minutes after probe injection. Images of ¹⁸F-ZD-G2 probe and ¹⁸FDG were compared.

FIG. 19 shows representative antibody and probe staining of in vitro human samples. 5 patients were evaluated-2 with WHO group I (IPAH) and 3 with WHO group III. Average mean PA pressure was 39 mm Hg, mean PVR was 8 WU, and mean TPG was 25 mm Hg. Both WHO group I patients were on epoprostenol.

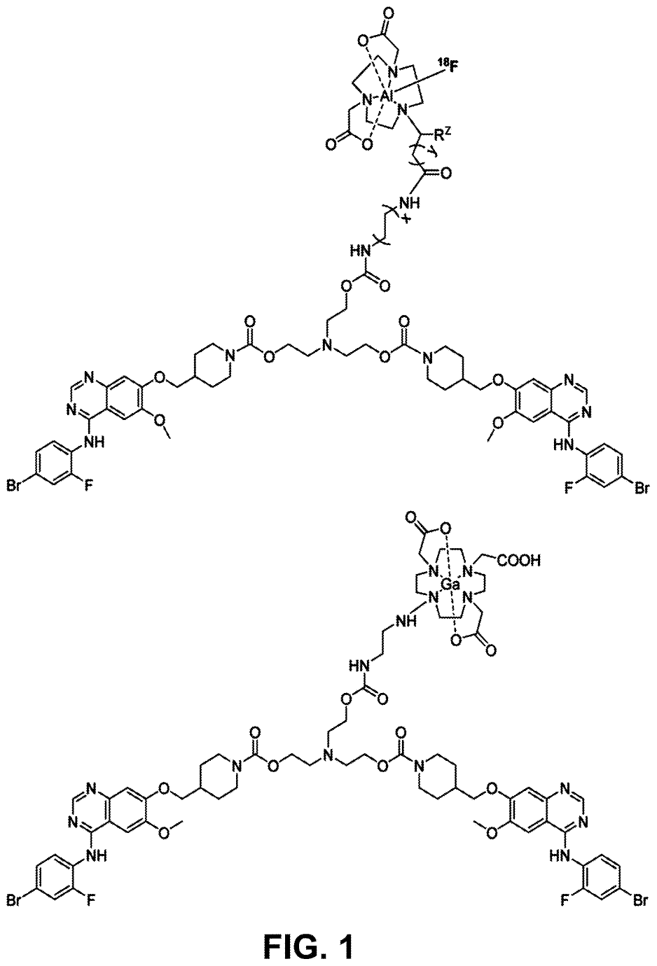

### DETAILED DESCRIPTION

The following description of the disclosure is provided as an enable teaching of the disclosure in its best, currently known embodiments. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are part of the present disclosure. Thus, the following description is provided as illustrative of the principles of the present disclosure and not in limitation thereof.

### DEFINITIONS

Before the present compounds, compositions, and methods are disclosed and described, it is to be understood that this invention is not limited to specific synthetic methods, or specific route of administration, as such may, of course, vary. It is also to be understood that the terminology used herein is for the purpose of describing particular embodiments only and is not intended to be limiting.

Throughout this application, various publications are referenced. The disclosures of these publications in their entireties are hereby incorporated by reference into this application in order to more fully describe the state of the art to which this invention pertains.

As used in the specification and the appended claims, the singular forms “a,” “an,” and “the” include plural referents unless the context clearly dictates otherwise. Thus, for example, reference to “a compound” includes mixtures of such compounds and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. Unless stated to the contrary, the term “about” means within 5%, e.g., within 1, 2, 3, or 4% of the stated value, or less.

In this specification and in the claims which follow, reference will be made to a number of terms which shall be defined to have the following meanings:

“Optional” or “optionally” means that the subsequently described event or circumstance does or does not occur, and that the description includes instances where said event or circumstance occurs and instances where it does not. For example, the phrase “optionally substituted alkyl” means that the alkyl group is or is not substituted and that the description includes both unsubstituted lower alkyl and lower alkyl where there is substitution.

“Pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio. Pharmaceutically acceptable materials are known to those of ordinary skill in the art.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

Terms for various chemical moieties as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆ alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Haloalkoxy” indicates a haloalkyl group as defined herein attached through an oxygen bridge (oxygen of an alcohol radical).

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or uccal. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 3, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfone, ethane disulfonic, oxalic, isethionic, HOOC—(CH₂)₁₋₄—COOH), and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Methods of Diagnosis and Treatment

The present disclosure provides methods for the detection of a disease associated with pulmonary vascular remodeling (such as pulmonary hypertension) using small molecular PET imaging agents that bind to vascular endothelial growth factor receptor 2 (VEGFR-2). The present methods allow earlier detection of such diseases compared to present methods (such as echocardiography or cardiac catheterization) due to detection being tied to the pathophysiological mechanism for the disease instead of detecting late stage symptoms (such as increased arterial pressures). Further, methods of treatment are also provided that use detection of a disease associated with pulmonary vascular remodeling to determine the point when a therapy for the treatment of the disease is administered.

Thus, in one aspect, a method of detecting a disease associated with pulmonary vascular remodeling in a subject is provided, the method comprising:
(a) administering to the subject a compound of Formula I or Formula II

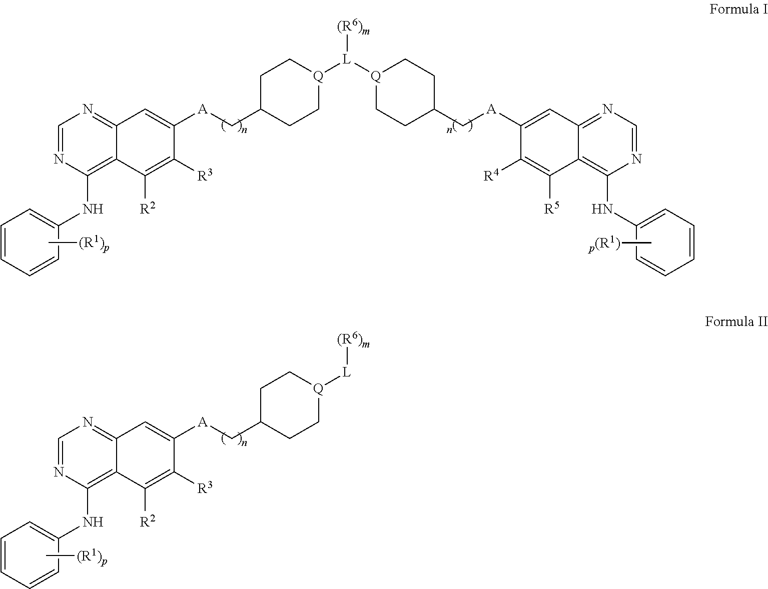

or a pharmaceutically acceptable salt thereof; and
(b) imaging the compound in the subject using positron emission tomography (PET) to detect uptake of the compound in a pulmonary vasculature of the subject;
wherein uptake of the compound in the pulmonary vasculature reflects vascular remodeling;
and wherein:
R¹ is independently selected at each occurrence from hydroxyl, halogen, C₁₋₃ alkyl, C₁₋₃ alkoxyl, C₁₋₃ alkanoyloxyl, trifluoromethyl, cyano, amino, and nitro;
R², R³, R⁴, and R⁵ are independently selected from hydrogen, hydroxyl, halogen, nitro, trifluoromethyl, cyano, C₁₋₃ alkyl, C₁₋₃ alkoxyl, C₁₋₃ alkylthio, and —NR⁷R⁸,
R⁷ and R⁸ are independently at each occurrence from hydrogen and C₁₋₃ alkyl;
A is —O—, —CH₂—, —S—, —SO—, —SO₂—, —NR⁷CO—, —CONR⁷—, —SO₂NR⁷—, —NR⁷SO₂—, or —NR⁷—;
Q is N or CH;
L is a linker;
R⁶ contains a positron emission tomography (PET) detectable moiety;
n is an integer from 1 to 5, for example, n can be 1, 2, 3, 4, or 5;
m is an integer from 1 to 5, for example, m can be 1, 2, 3, 4, or 5; and
p is an integer from 1 to 4, for example p can be 1, 2, 3, or 4.

The compounds of Formula I and Formula II have one or more VEGFR-2 binding moieties having the chemical structure of Formula A:

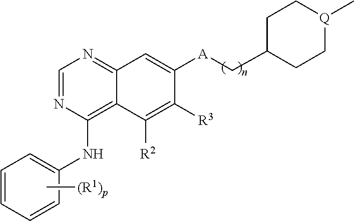

wherein all variables are as defined herein.

In a preferred aspect of the VEGFR-2 binding moiety, R¹ can be a halogen, for example, Br or F. In other examples, R¹ can be hydroxyl, C₁₋₃ alkyl, or C₁₋₃ alkyl.

In other examples R² is preferably hydrogen. In still other examples, R³ is preferably C₁₋₃ alkyl, or C₁₋₃ alkyl. In yet further examples, A is preferably —O— and Q is preferably N.

Also, in the disclosed compounds n is preferably 1 or 2 and p is preferably 1 or 2.

In some aspects, the compound is of Formula III, which corresponds to Formula I wherein A is —O—, Q is N, n is 1, and R² and R⁵ are both hydrogen:

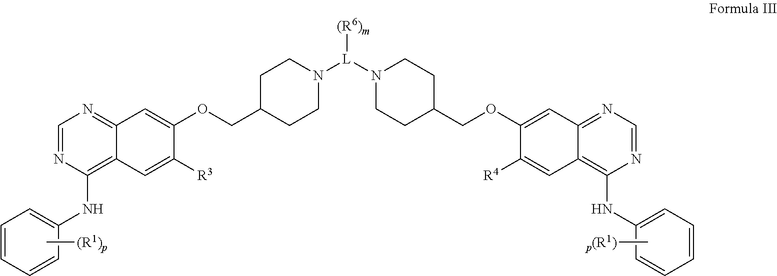

wherein:
R¹ can be hydroxyl, halogen, C₁₋₃ alkyl, C₁₋₃ alkoxyl, C₁₋₃ alkanoyloxyl, trifluoromethyl, cyano, amino or nitro;
R³ and R⁴ can be, independent of one another, hydrogen, hydroxyl, halogen, nitro, trifluoromethyl, cyano, C₁₋₃ alkyl, C₁₋₃ alkoxyl, C₁₋₃ alkylthio, or —NR⁷R⁸, wherein R⁷ and R⁸, which can be the same or different, each represents hydrogen or C₁₋₃ alkyl;
L is a linker;
R⁶ contains a PET detectable moiety;
m is an integer from 1 to 5, for example, m can be 1, 2, 3, 4, or 5; and
p is an integer from 1 to 4, for example 1, 2, 3, or 4.

In some aspects, the compounds have Formula IV:

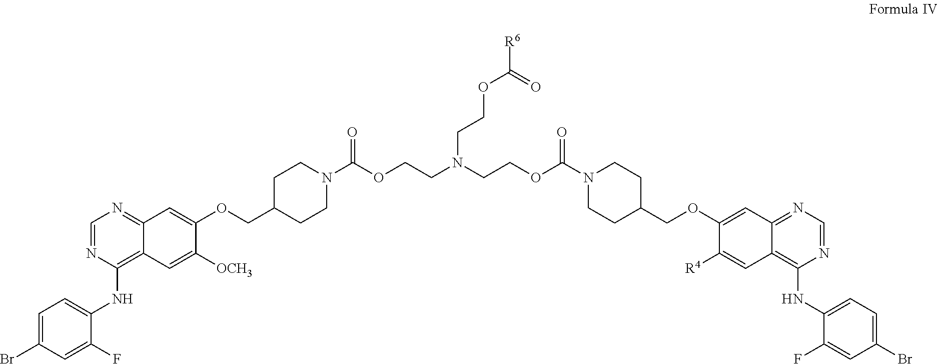

wherein R⁶ contains a PET detectable moiety.

In some aspects, the compound can have a chemical formula of Formula V, which corresponds to Formula II wherein A is —O—, Q is N, R² is hydrogen, and n is 1:

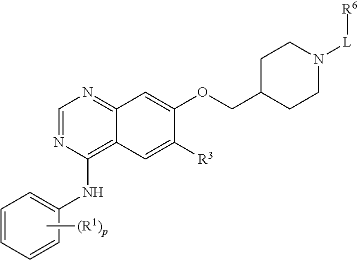

wherein:
R¹ can be hydroxyl, halogen, C₁₋₃ alkyl, C₁₋₃ alkoxyl, C₁₋₃ alkanoyloxyl, trifluoromethyl, cyano, amino, or nitro;
R³ can be hydrogen, hydroxy, halogen, nitro, trifluoromethyl, cyano, C₁₋₃ alkyl, C₁₋₃ alkoxy, C₁₋₃ alkylthio, or —NR⁷R⁸, wherein R⁷ and R⁸, which can be the same or different, each represents hydrogen or C₁₋₃ alkyl;
L is a linker;
R⁶ contains a PET detectable moiety; and
p is an integer from 1 to 4, for example, p can be 1, 2, 3, or 4.

In some aspects, the compounds have the following structure:

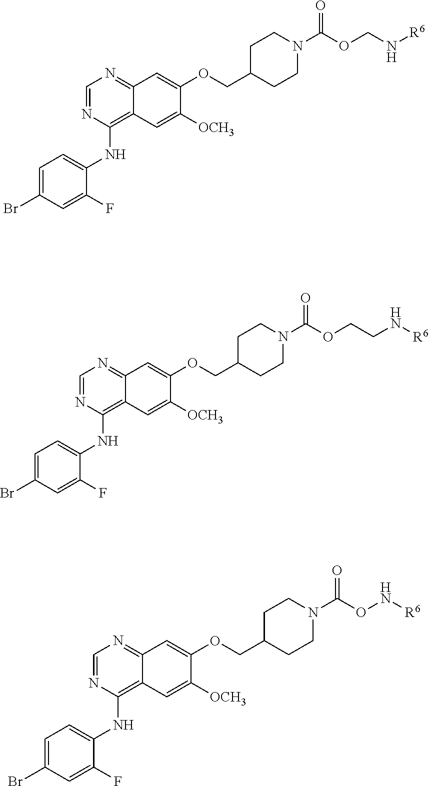

wherein R⁶ contains PET detectable moiety.

The VEGFR-2 binding moiety and the detectable moiety on the compounds are linked via a linker. The term “linker”, as used herein, refers to one or more polyfunctional, e.g. bifunctional molecules or trifunctional molecules, which can be used to covalently couple the VEGFR-2 binding moiety and the detectable moiety and which do not interfere with the properties of the VEGFR-2 binding moiety and the detectable moiety in the compounds. The VEGFR-2 binding moiety and the detectable moiety can be attached to any part of the linker. The linker can be attached to any part of the VEGFR-2 binding moiety and the detectable moiety.

In some aspects, the linker is flexible. In some aspects, the linker is stable and biocompatible. In some aspects, the covalent bond formed between the linker and the VEGFR-2 binding moiety and/or the detectable moiety is stable. Stable, as used herein refers to a covalent bond that remains at least 70%, preferably at least 80%, more preferably at least 90% intact in aqueous solution at temperatures ranging from about 0 °C to about 100 °C, at a pH ranging from about 2 to about 12, for at least 1 hour. The covalent bond formed between the linker and the VEGFR-2 binding moiety and/or the detectable moiety is hydrolytically and reductively stable.

The linker can be a single atom, such as a heteroatom (e.g., O, N, or S), a group of atoms, such as a functional group (e.g., amine, —C(═O)—, —CH₂—), or multiple groups of atoms, such as an alkylene chain. Suitable linkers include but are not limited to oxygen, sulfur, carbon, nitrogen, alkyl, alkenyl, cycloalkyl, cycloalkenyl, heterocycloalkyl, alkoxyl, aryl, heteroaryl, ether, amine, diamine, amide, alkylamine, thioether, carboxylates, polymer, derivatives or combinations thereof.

The linker can be R¹⁴, C(O)R¹⁴C(O), C(O)OR¹⁴OC(O), C(O)R¹⁴N, C(O)OR¹⁴NH, NHR¹⁴NH, or C(O)NHR¹⁴NHC(O), C(S)OR¹⁴OC(S); wherein R¹⁴ is O, S, C₁-C₂₀ alkyl; C₁-C₂₀ heteroalkyl; C₁-C₂₀ alkylamine; C₁-C₂₀ alkoxyl; C₁-C₂₀ alkanoyloxyl; or C₁-C₂₀ alkylamido, any of which can be optionally substituted with one or more substituents including halogen, alkoxyl, alkyl, alkenyl, cycloalkyl, cycloalkenyl, heterocycloalkyl, aryl, heteroaryl, amine, cyano, nitro, hydroxyl, carbonyl, acyl, carboxylic acid (—COOH), —C(O)R¹², —C(O)OR¹², carboxylate (—COO—), primary amide (e.g., —CONH₂), secondary amide (e.g., —CONHR¹²), —C(O)NR¹²R¹³, —NR¹²R¹³, —NR¹²S(O)₂R¹³, —NR¹²C(O)R¹³, —S(O)₂R¹², —SR¹², and —S(0)₂NR¹²R¹³, sulfinyl group (e.g., —SOR¹²), and sulfonyl group (e.g., —SOOR¹²);
wherein R¹² and R¹³ can each independently be hydrogen, halogen, hydroxyl, alkyl, haloalkyl, alkenyl, alkynyl, cycloalkyl, heterocycloalkyl, aryl, heteroaryl, carbonyl, cyano, amino, alkylamino, dialkylamino, alkoxyl, aryloxyl, cycloalkyl, alkoxycarbonyl, aminocarbonyl, alkylaminocarbonyl, or dialkylaminocarbonyl.

In some aspects, the linker is NR¹⁴R¹⁵R¹⁶ or (CH)R¹⁴R¹⁵R¹⁶, wherein the VEGFR-2 binding moiety or detectable moiety are bonded to at least one of R¹⁴R¹⁵R¹⁶, and wherein R¹⁴, R¹⁵, and R¹⁶ are each independently hydrogen, C₁-C₂₀ alkyl; C₁-C₂₀ heteroalkyl; C₁-C₂₀ alkylamine; C₁-C₂₀ alkoxy; C₁-C₂₀ alkanoyloxy; or C₁-C₂₀ alkylamido; any of which can be optionally substituted with one or more substituents independently selected from the group consisting of halogen; hydroxyl; cyano; nitro; amino; alkylamino; dialkylamino; amido; alkylamido; ═O; —S(O)₂; —SO—; —S—; —S(O)₂N—; haloalkyl; hydroxyalkyl; carboxy; alkoxy; aryloxy; alkoxycarbonyl; aminocarbonyl; alkylaminocarbonyl; and dialkylaminocarbonyl. For example, the linker is —(C(O)R¹⁴)₃N, —(R¹⁴)₃N, —(S(O)₂R¹⁴)₃N, —(C(O)R¹⁴)₃CH, —(R¹⁴)₃CH, or —(S(O)₂R¹⁴)₃CH. In some aspects, C₁₋₂₀ refers to alkyl groups containing 1, 2, 3, 4, 5, 6, 7, 8, 9, 10, 11, 12, 13, 14, 15, 16, 17, 18, 19, or 20 carbons.

In some aspects, the linker is —(CO—R¹⁴)₂NH, —(R¹⁴)₂NH, —(SO₂R¹⁴)₂NH, —(SOR¹⁴)₂NH, —(OR¹⁴)₂NH, —(O—CO—R¹⁴)₂NH, —(CO—O—R¹⁴)₂NH, —(CO—R¹⁴)₂CH₂, —(R¹⁴)₂CH₂, —(SO₂R¹⁴)₂CH₂, —(SOR¹⁴)₂CH₂, —(O—CO—R¹⁴)₂CH₂, or —(OR¹⁴)₂CH₂.

In some aspects, the linker is —(CO—R¹⁴)₃N, —(R¹⁴)₃N, —(SO₂R¹⁴)₃N, —(SOR¹⁴)₃N, —(OR¹⁴)₃N, —(O—CO—R¹⁴)₃N, —(CO—O—R¹⁴)₃N, —(CO—R¹⁴)₃CH, —(R¹⁴)³CH, —(SO₂R¹⁴)₃CH, —(SOR¹⁴)₃CH, —(O—CO—R¹⁴)₃CH, or —(OR¹⁴)₃CH.

In some aspects of Formula III, the linker can be selected from —(CO—(C₂-C₄ alkyl))₃N, —(C₂-C₄ alkyl)₃N, —(SO₂—(C₂-C₄ alkyl))₃N, —(SO—(C₂-C₄ alkyl))₃N, —(O—(C₂-C₄ alkyl))₃N, —(O—CO—(C₂-C₄ alkyl))₃N, —(CO—O—(C₂-C₄ alkyl))₃N, —(CO—(C₂-C₄ alkyl))₃CH, —(C₂-C₄ alkyl)₃CH, —(SO₂—(C₂-C₄ alkyl))₃CH, —(SO—(C₂-C₄ alkyl))₃CH, —(O—CO—(C₂-C₄ alkyl))₃CH, or —(O—(C₂-C₄ alkyl))₃CH.

In some aspects of Formula III, the linker can be

In some aspects of Formula III, the linker can be —(CO—O—R¹⁴)₃N or —(CO—O—R¹⁴)₃CH, wherein R¹⁴ is a C₂₋₄ alkyl.

In some aspects of Formula III, the linker can be —(CO—O-(independently C₂-C₄ alkyl))₃N.

In some aspects of Formula III, the linker can be

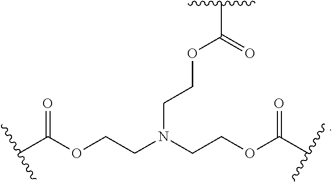 [SMILES: *C(=O)OCCN(CCOC(*)=O)CCOC(*)=O]

In some aspects, the linker can be an amino acid. The amino acid can be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Examples of suitable amino acids include, but are not limited to, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, a derivative, or combinations thereof.

In some aspects, the linker is an amino dicarboxylic acid. In some aspects, the amino dicarboxylic acid can have from 2 to 30 carbon atoms. Examples of suitable amino dicarboxylic acids include, but are not limited to, 1,6-dicarboxylic-2-amino hexanoic acid, 1,7-dicarboxylic-2-amino heptanoic acid, 1,8-dicarboxylic-2-amino octanoic acid, α-aminosuccinic acid, β-aminoglutaric acid, β-aminosebacic acid, 2,6-piperidine dicarboxylic acid, 2,5-pyrrole dicarboxylic acid, 2-carboxypyrrole-5-acetic acid, 2-carboxypiperidine-6-propionic acid, 2-aminoadipic acid, 3-aminoadipic acid, α-aminoazelaic acid, and 4-aminobenzene-1,3-dicarboxylic acid.

In some aspects, the linker can be a dicarboxylic acid. In some aspects, the dicarboxylic acid can have from 2 to 20 carbon atoms. Examples of dicarboxylic acid include, but are not limited to, butanedioic acid, pentanedioic acid, hexanedioic acid, heptanedioic acid, octanedioic acid, nonanedioic acid, decanedioic acid, undecanedioic acid, dodecanedioic acid, tridecanedioica acid, 1,12-dodecanedicarboxylic acid, 1,15-pentadecanedicarboxylic acid, hexadecanedioic acid, and 1,15-pentadecanedicarboxylic acid. In some aspects, the dicarboxylic acid is an halogenated dicarboxylic acid, hydroxy dicarboxylic acid, or ether dicarboxylic acid.

In some aspects, the linker can be a tricarboxylic acid or a derivative thereof. In some aspects, the tricarboxylic acid can have from 2 to 30 carbon atoms. The tricarboxylic acid can be aliphatic or cyclic. Examples of tricarboxylic acid include, but are not limited to, 2-phosphonobutane-1,2,4-tricarboxylic acid and 1,2,3-propane tricarboxylic acid.

In some aspects, the linker can be an alcohol or a derivative thereof. The alcohol can be a diol, triol, amino alcohol, amino dialcohol, amino trialcohol, ethylene glycol, propylene glycol, or a derivative. In some aspects, the alcohol can have from 2 to 30 carbon atoms. Examples of suitable alcohols include, but are not limited to, triethanolamine, 2-aminoethanol, diisopropanolamine, triisopropanolamine, amino hexanol, 2-[(2-methoxyethyl)methylamino]-ethanol, propanolamine, N-methylethanolamine, diethanolamine, butanol amine, isobutanolamine, pentanol amine, 1-amino-3-(2-methoxyethoxy)-2-propanol, 2-methyl-4-(methylamino)-2-butanol, 6-amino-1-hexanol, heptaminol, isoetarine, norepinephrine, sphingosine, phenylpropanolamine, derivatives, and combinations thereof.

In other aspects, the linker can be a polymer. A wide variety of polymers and methods for forming the polymers are known in the art of polymer science. Polymers can be degradable or non-degradable polymers. Polymers can be natural or unnatural (synthetic) polymers. Polymers can be homopolymers or copolymers comprising two or more monomers. In terms of sequence, copolymers can be random, block, or comprise a combination of random and block sequences. The polymers can in some aspects be linear polymers, branched polymers, or hyperbranched/dendritic polymers. The polymers can also be present as a crosslinked particle or surface functionalized inorganic particle. Suitable polymers include, but are not limited to poly(vinyl acetate), copolymers of styrene and alkyl acrylates, and copolymers of vinyl acetate and acrylic acid, polyvinylpyrrolidone, dextran, carboxymethylcellulose, polyethylene glycol, polyalkylene, polyanhydrides, poly (ester anhydrides), polyhydroxy acids, such as polylactide (PLA), polyglycolide (PGA), poly (lactide-co-glycolide) (PLGA), poly-3-hydroxybutyrate (PHB), poly-4-hydroxybutyrate (P4HB), polycaprolactone, polyacrylates and polymethacrylates; polyanhydrides; polyorthoesters; polysytyrene (PS), poly(ethylene-co-maleic anhydride), poly(ethylene maleic anhydride-co-L-dopamine), poly(ethylene maleic anhydride-co-phenylalanine), poly (ethylene maleic anhydride-co-tyrosine), poly(butadiene-co-maleic anhydride), poly(butadiene maleic anhydride-co-L-dopamine) (pBMAD), poly(butadiene maleic anhydride-co-phenylalanine), poly(butadiene maleic anhydride-co-tyrosine), poly(bis carboxy phenoxy propane-co-sebacic anhydride) (poly (CCP:SA)), alginate; and poly(fumaric anhydride-co-sebacic anhydride (p[FA:SA]), copolymers of p[FA:SA], polyacrylates, and polyacrylamides, and copolymers thereof, and combinations thereof.

Other suitable linkers include, but are not limited to, diamino compounds such as ethylenediamine, 1,2-propylenediamine, 1,5-pentanediamine, 1,6-hexanediamine, and the like.

The compounds as used in the present methods contain a PET detectable moiety. In some aspects, the PET detectable moiety is an ¹⁸F radiolabeled moiety or a ⁶⁸Ga radiolabeled moiety. In some aspects, the PET detectable moiety is biocompatible. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

In some aspects, the PET detectable moiety is an ¹⁸F radiolabeled moiety. In some aspects, the PET detectable moiety is selected from:

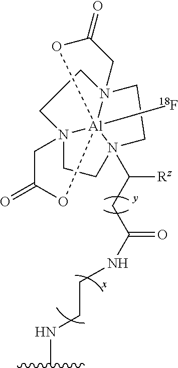

wherein:
x is 1 or 2;
y is 0 or 1; and
R^{z} is H or COOH.

In some aspects, the compound is of Formula A

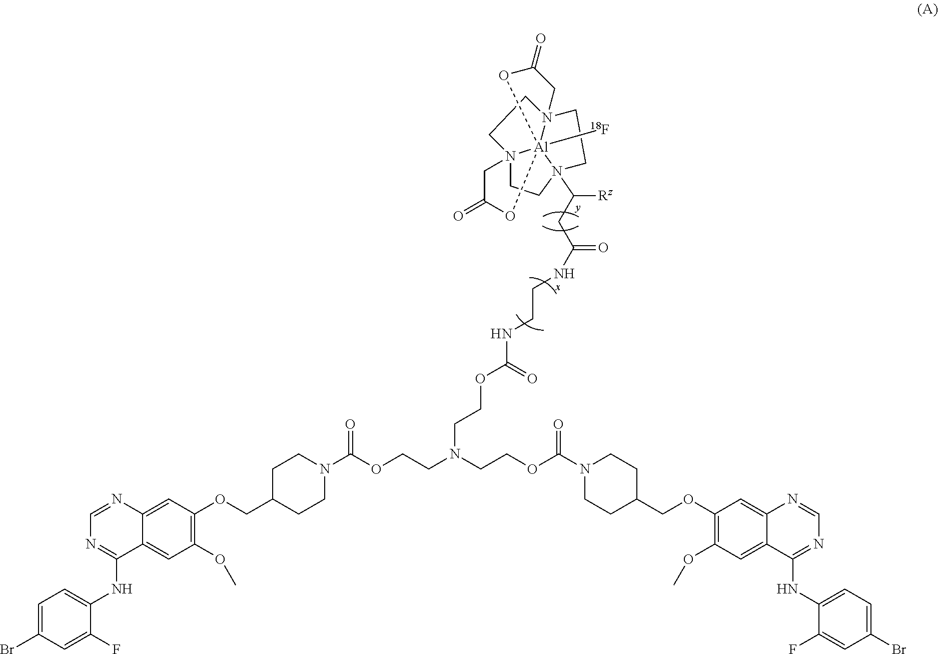

or a pharmaceutically acceptable salt thereof;
wherein:
x is 1 or 2;
y is 0 or 1; and
R^{z} is H or COOH.

In some aspects of Formula A, x is 1. In some aspects of Formula A, x is 2.

In some aspects of Formula A, y is 0. In some aspects of Formula A, y is 1.

In some aspects of Formula A, R^{z} is H. In some aspects of Formula A, R^{z} is COOH.
or a pharmaceutically acceptable salt thereof.

In some aspects of Formula A, the compound is selected from:

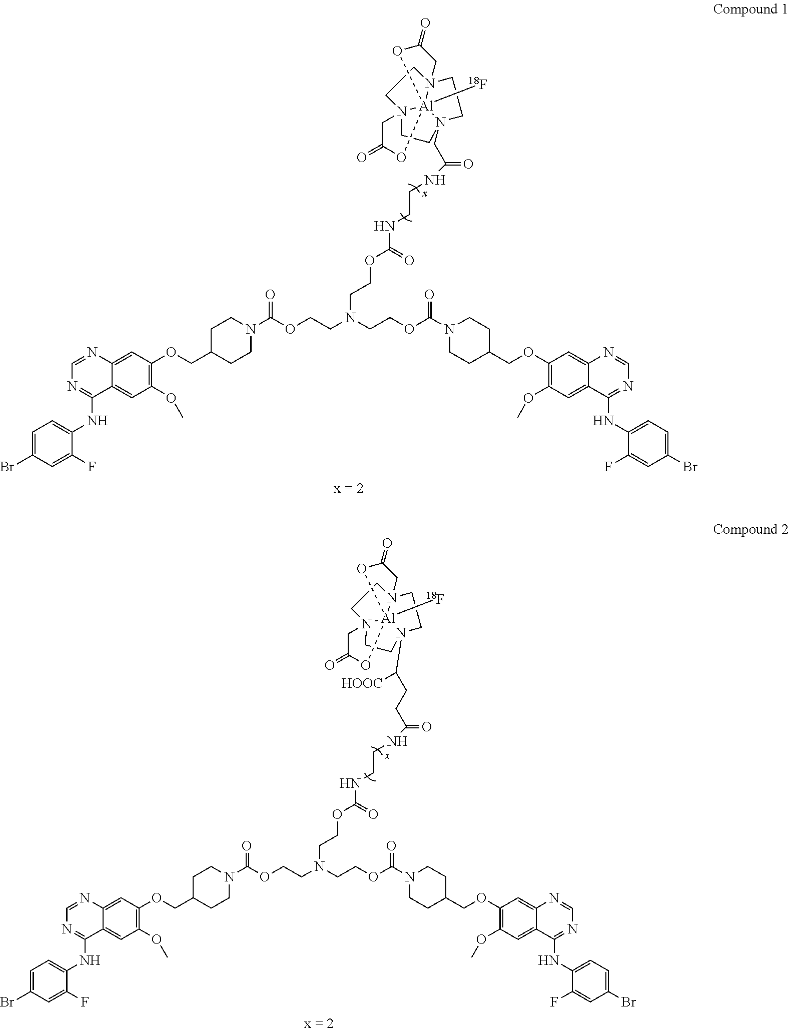

In another aspect, a compound is provided of Formula A, or a pharmaceutically acceptable salt thereof. In some aspects, the compound is Compound 1 or Compound 2.

In some aspects, the PET detectable moiety is a ⁶⁸Ga radiolabeled moiety. In some aspects, the PET detectable moiety can be:

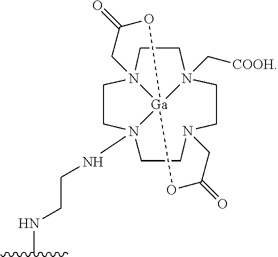

In some aspects, the compound is Compound 3:

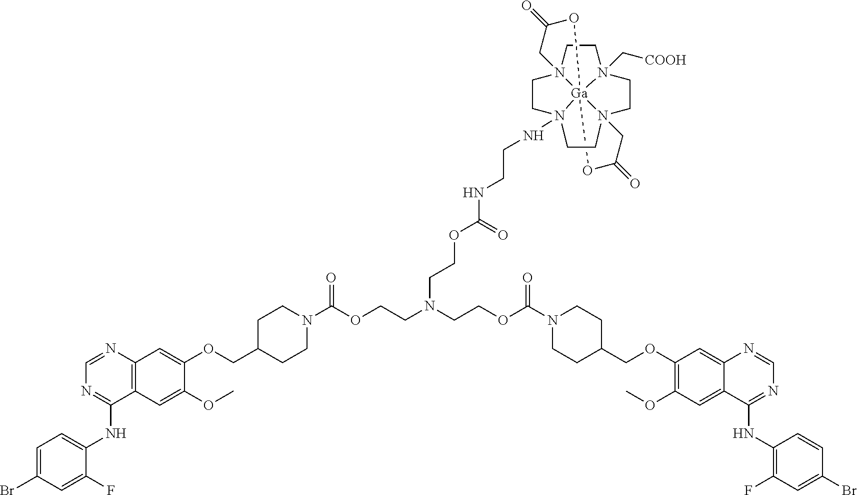

or a pharmaceutically acceptable salt thereof.

In some aspects, the disease associated with pulmonary vascular remodeling is pulmonary hypertension. Pulmonary hypertension (PH or PHTN) is a condition of increased blood pressure within the arteries of the lungs. A patient is deemed to have pulmonary hypertension if the pulmonary mean arterial pressure is greater than 23 mmHg at rest, or greater than 30 mmHg during exercise.

There are 5 groups of pulmonary hypertension according to WHO classification, with the most recent system being as follows:

WHO Group I comprises disorders associated with pulmonary arterial hypertension (PAH). PAH may be idiopathic, due to a heritable condition (such as BMPR2, ALK1, SMAD9, caveolin 1, or KCNK3 mutations), drug- or toxin-induced (such as the result of methamphetamine use), or may be associated with other disorders such as connective tissue disease, HIV infection, portal hypertension, congenital heart diseases, or schistosomiasis.

WHO Group I may be further divided in WHO Group I′ and WHO Group I″. WHO Group I′ comprises pulmonary veno-occlusive disease (PVOD) and pulmonary capillary hemangiomatosis (PCH). PVOD is a rare form of pulmonary hypertension caused by progressive blockage of the small veins in the lungs. The blockage leads to high blood pressures in the arteries of the lungs which, in turn, leads to heart failure. PCH is a disease affective the blood vessels of the lungs, where abnormal capillary proliferation and venous fibrous intimal thickening result in progressive increase in vascular resistance. PH categorized in WHO Group I′ may be idiopathic, the result of a heritable conditions (such as EIF2AK4 mutations), drug-, toxin-, or radiation-induced, or associated with other conditions such as connective tissue disease or HIV infection. WHO Group I″ comprises persistent pulmonary hypertension of the newborn.

WHO Group II comprises pulmonary hypertension secondary to left heart disease, such as left ventricular systolic dysfunction, left ventricular diastolic dysfunction, valvular heart disease, congenital/acquired left heart inflow/outflow tract obstruction and congenital cardiomyopathy, and congenital/acquired pulmonary venous stenosis.

WHO Group III comprises pulmonary hypertension due to lung disease or chronic hypoxia. WHO Group III PH may be the result of chronic obstructive pulmonary disease (COPD), interstitial lung disease, mixed restrictive and obstructive pattern pulmonary diseases, sleep-disordered breathing, alveolar hypoventilation disorders, chronic exposure to high altitude, and developmental abnormalities.

WHO Group IV comprises pulmonary hypertension due to chronic arterial obstruction. WHO Group IV PH may comprise chronic thromboembolic pulmonary hypertension (CTEPH). Alternatively, it may be the result of other pulmonary artery obstructions such as those resulting from angiosarcoma or other tumor within the blood vessels, arteritis, congenital pulmonary artery stenosis, or parasitic infection (such as hydatidosis).

WHO Group V comprises pulmonary hypertension with unclear or multifactorial mechanisms. PH in this group may be the result of hematologic diseases (such as chronic hemolytic anemia including sickle cell disease), systemic diseases (such as sarcoidosis, pulmonary Langerhans cell histiocytosis, lymphangioleiomyomatosis, neurofibromatosis, or vasculitis), metabolic disorders (such as glycogen storage disease, Gaucher disease, or thyroid diseases), or others (such as pulmonary tumoral thrombotic microangiopathy, fibrosing mediastinitis, chronic kidney failure, or segmental pulmonary hypertension).

In some aspects, the compounds as used herein accumulate in the pulmonary vasculature if vascular remodeling is and/or has previously occurred. In some aspects, the compounds as used herein can accumulate in the pulmonary vasculature if vascular remodeling is and/or has previously occurred within about 48 hours, about 36 hours, about 24 hours, about 23 hours, about 22 hours, about 21 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, about 1.5 hours, about 1 hour, about 50 minutes, about 45 minutes, about 40 minutes, about 35 minutes, about 30 minutes, about 25 minutes, about 20 minutes, about 15 minutes, about 10 minutes, or about 5 minutes post administration.

In some aspects, vascular remodeling is determined to be occurring and/or to have occurred when the pulmonary vasculature exhibits sufficient uptake of the compounds used herein post administration. Sufficient, as used herein, refers to uptake of the disclosed compounds into the pulmonary vasculature such that imaging of the vasculature exhibits low background noise. In some aspects, the pulmonary vasculature exhibits uptake of greater than about 5% ID/g, greater than about 4.7% ID/g, greater than about 4.5% ID/g, greater than about 4.3% ID/g, greater than about 4% ID/g, greater than about 3.7% ID/g, greater than about 3.5% ID/g, greater than about 3.3% ID/g, greater than about 3% ID/g, greater than about 2.7% ID/g, greater than about 2.5% ID/g, greater than about 2.3% ID/g, greater than about 2% ID/g, greater than about 1.8% ID/g, greater than about 1.5% ID/g, greater than about 1.3% ID/g, greater than about 1% ID/g, greater than about 0.9% ID/g, greater than about 0.8% ID/g, greater than about 0.6% ID/g, greater than about 0.5% ID/g, greater than about 0.4% ID/g, greater than about 0.3% ID/g, greater than about 0.2% ID/g, greater than about 0.1% ID/g, for example about 2.7% ID/g, about 3.70% ID/g, about 3.8% ID/g, about 0.3% ID/g, about 0.7% ID/g, or about 0.5% ID/g of the disclosed compounds post administration.

In some aspects, the pulmonary vasculature exhibits a standard uptake value (SUV) ranging from about 2 to about 10, for example 2.5, 3.0, 3.5, 4.0, 4.5, 5.0 5.5, 6.0, 6.5, 7.0, 7.5, 8.0, 8.5, 9.0, or 9.5. In some aspects, the pulmonary vasculature exhibits a SUV of 2 or greater, 2.5 or greater, 3 or greater, 3.5 or greater, 4 or greater, 4.5 or greater, 5 or greater, 4.5 or greater, 5 or greater, 5.5 or greater, 6 or greater, 6.5 or greater, 7 or greater, 7.5 or greater, 8 or greater, 8.5 or greater, 9 or greater, 9.5 or greater, or 10 or greater.

25 In some aspects, non-targeted tissues, such as the kidney, muscles, heart, blood, lung, gastrointestinal tract, and/or spleen exhibit low uptake of the compounds used herein. In some aspects, uptake of the compounds used herein in the non-targeted tissues is less than about 2.5% ID/g, less than about 2.3% ID/g, less than about 2% ID/g, less than about 1.8% ID/g, less than about 1.5% ID/g, less than about 1.3% ID/g, less than about 1% ID/g, less than about 0.9% ID/g, less than about 0.8% ID/g, less than about 0.6% ID/g, less than about 0.5% ID/g, less than about 0.4% ID/g, less than about 0.3% ID/g, less than about 0.2% ID/g, or less than about 0.1% ID/g at about 24 hours, about 23 hours, about 22 hours, about 20 hours, about 19 hours, about 18 hours, about 17 hours, about 16 hours, about 15 hours, about 14 hours, about 13 hours, about 12 hours, about 11 hours, about 10, about 9 hours, about 8 hours, about 7 hours, about 6 hours, about 5 hours, about 4 hours, about 3 hours, about 2 hours, about 1.5 hour, about 1 hour, about 50 minutes, about 45 minutes, about 40 minutes, about 35 minutes, about 30 minutes, about 25 minutes, about 20 minutes, about 15 minutes, about 10 minutes, or about 5 minutes post administration.

In some aspects, the ratio of compound uptake in the pulmonary vasculature to non-targeted tissue can be high. In some aspects, the ratio of compound uptake in the pulmonary vasculature to non-targeted tissue can be greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 25, greater than 30, greater than 35, greater than 40, greater than 45, or greater than 50. In some aspects, the ratio of compound uptake in the pulmonary vasculature to non-targeted tissue can remain high for as long as about 2 hours, about 3 hours, about 4 hours, about 6 hours, about 10 hours, about 15 hours, about 18 hours, about 20 hours, about 24 hours, about 36 hours, about 46 hours, or as long as the compound is in a subject, post administration.

In another aspect, a method of treating a disease associated with pulmonary vascular remodeling in a subject is provided, the method comprising:
(a) administering to the subject a compound of Formula I or Formula II, or a pharmaceutically acceptable salt thereof;
(b) imaging the compound in the subject using positron emission tomography (PET) to detect uptake of the compound in a pulmonary vasculature of the subject; and
if uptake of the compound is detected in the pulmonary vasculature in (b), then (c) administering to the subject a therapy for the disease associated with pulmonary vascular remodeling.

In some aspects, the therapy may comprise surgery. In some aspects, the surgery may comprise atrial septostomy. Atrial septostomy is a surgical procedure that creates a communication between the right and left atria of the heart. It relieves pressure on the right side of the heart, but at the cost of lower oxygen levels in the blood. In some aspects, the surgery may comprise pulmonary thromboendarterectomy (PTE). PTE is a surgical procedure that is used for chronic thromboembolic pulmonary hypertension that comprises the removal of an organized thrombus along with the lining of the pulmonary artery.

In some aspects, the therapy may comprise one or more additional therapeutic agents. Representative examples of additional therapeutic agents which may be administered include, but are not limited to, a vasodilator, a guanylate cyclase activator, an endothelin receptor antagonist, a phosphodiesterase type 5 inhibitor, a calcium channel blocker, an anticoagulant, digoxin, or a diuretic.

Representative examples of vasodilators which may be used include, but are not limited to, epoprostenol, Treprostinil, and iloprost.

Representative examples of guanylate cyclase activators which may be used include, but are not limited to, cinaciguat and riociguat.

Representative examples of endothelin receptor antagonists which may be used include, but are not limited to, sitaxentan, ambrisentan, atrasentan, BQ-123, zibotentan, bosentan, mcitentan, tezosentan, BQ-788, and A192621.

Representative examples of phosphodiesterase type 5 inhibitor which may be used include, but are not limited to, sildenafil and tadalafil.

Representative examples of calcium channel blockers which may be used include, but are not limited to, amlodipine, diltiazem, and nifedipine.

### Methods of Administration

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the diagnosis or treatment of a disease associated with pulmonary vascular remodeling, for example pulmonary hypertension, in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain aspects, the preservative is an anti-oxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl uccal i, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, uccal, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, buccal ions, buccal gums, including xanthan gum, guar gum, gum buccal, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy (Polyethylene glycol)-1000], 1,2-Distearoyl-sn-glycero-3-Phosphoethanolamine-N-[Methoxy (Polyethylene glycol)-2000], and 1,2-Distearoyl-sn-glycero-3-Phosphoethanolamine-N-[Methoxy (Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain aspects, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain aspects, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

In one aspect, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug occurs. The alveoli are covered by a thin epithelium without cilia or a mucus blanket and secrete surfactant phospholipids. Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, uccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

In another aspect, the following embodiments of the present disclosure are also provided:

Embodiment 1. A method of detecting a disease associated with pulmonary vascular remodeling in a subject, the method comprising:
(a) administering to the subject a compound of Formula I or Formula II

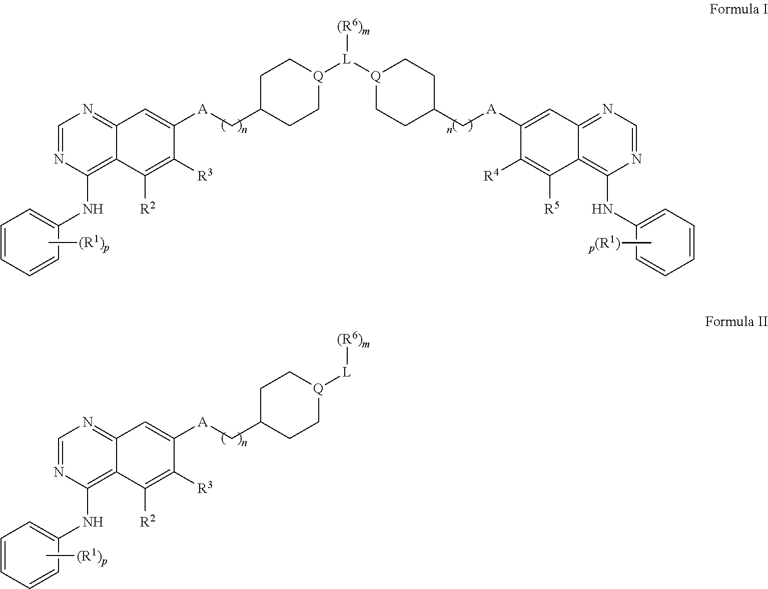

or a pharmaceutically acceptable salt thereof; and
(b) imaging the compound in lungs of the subject using positron emission tomography (PET) to determine uptake of the compound in a pulmonary vasculature of the lungs;
wherein retention of the compound in the pulmonary vasculature reflects vascular remodeling;
and wherein:
R¹ is independently selected at each occurrence from hydroxyl, halogen, C₁₋₃ alkyl, C₁₋₃ alkoxyl, C₁₋₃ alkanoyloxyl, trifluoromethyl, cyano, amino, and nitro;
R², R³, R⁴, and R⁵ are independently selected from hydrogen, hydroxyl, halogen, nitro, trifluoromethyl, cyano, C₁₋₃ alkyl, C₁₋₃ alkoxyl, C₁₋₃ alkylthio, and —NR⁷R⁸,
R⁷ and R⁸ are independently at each occurrence from hydrogen and C₁₋₃ alkyl;
A is —O—, —CH₂—, —S—, —SO—, —SO₂—, —NR⁷CO—, —CONR⁷—, —SO₂NR⁷—, —NR⁷SO₂—, or —NR⁷—;
Q is N or CH;
L is a linker;
R⁶ contains a positron emission tomography (PET) detectable moiety;
n is an integer from 1 to 5;
m is an integer from 1 to 5; and
p is an integer from 1 to 4.

Embodiment 2. The method of embodiment 1, wherein the PET detectable moiety is an ¹⁸F labeled moiety or ⁶⁸Ga labeled moiety.

Embodiment 3. The method of any one of embodiments 1 or 2, wherein the PET detectable moiety is selected from:

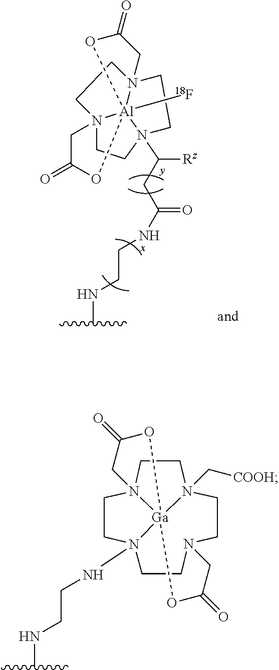

wherein:
x is 1 or 2;
y is 0 or 1; and
R^{Z} is H or COOH.

Embodiment 4. The method of any one of embodiments 1-3, wherein the compound is of Formula III

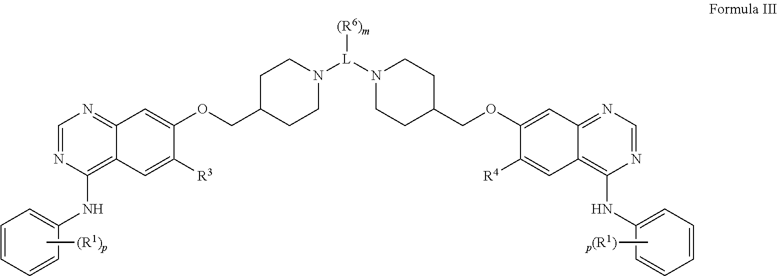

wherein:
R¹ is hydroxy, halogen, C₁₋₃ alkyl, C₁₋₃ alkoxyl, C₁₋₃ alkanoyloxyl, or trifluoromethyl; and
R³ and R⁴ are independently hydrogen, hydroxyl, halogen, C₁₋₃ alkyl, C₁₋₃ alkoxyl, or C₁₋₃ alkylthio.

Embodiment 5. The method of any one of embodiments 1-4, wherein the linker is —(CO—R¹⁴)₃N, —(R¹⁴)₃N, —(SO₂R¹⁴)₃N, —(SOR¹⁴)₃N, —(OR¹⁴)₃N, —(O—CO—R¹⁴)₃N, —(CO—O—R¹⁴)₃N, —(CO—R¹⁴)₃CH, —(R¹⁴)₃CH, —(SO₂R¹⁴)₃CH, —(SOR¹⁴)₃CH, —(O—CO—R¹⁴)₃CH, or —(OR¹⁴)₃CH.

Embodiment 6. The method of embodiment 5, wherein the linker is —(CO—O—R¹⁴)₃N or —(CO—O—R¹⁴)₃CH, wherein R¹⁴ is a C₂₋₄ alkyl.

Embodiment 7. The method of embodiment 5 or embodiment 6, wherein the linker is

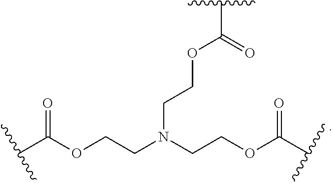 [SMILES: *C(=O)OCCN(CCOC(*)=O)CCOC(*)=O]

Embodiment 8. The method of any one of embodiments 1-7, wherein n is 1.

Embodiment 9. The method of any one of embodiments 1-8, wherein the compound is of Formula IV:

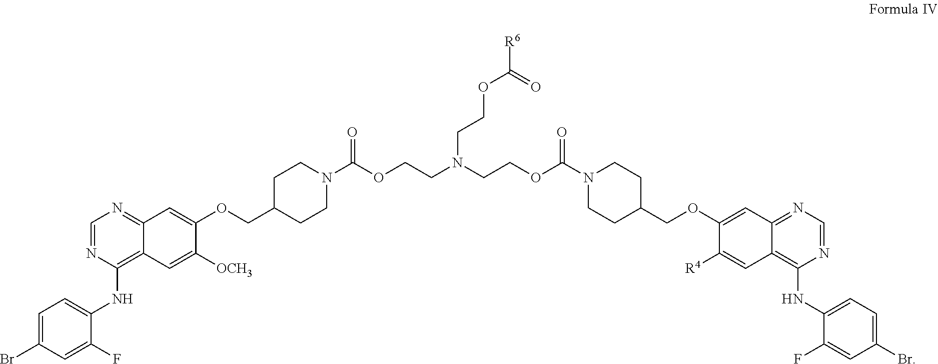

Embodiment 10. The method of any one of embodiments 1-9, wherein the compound is selected from:

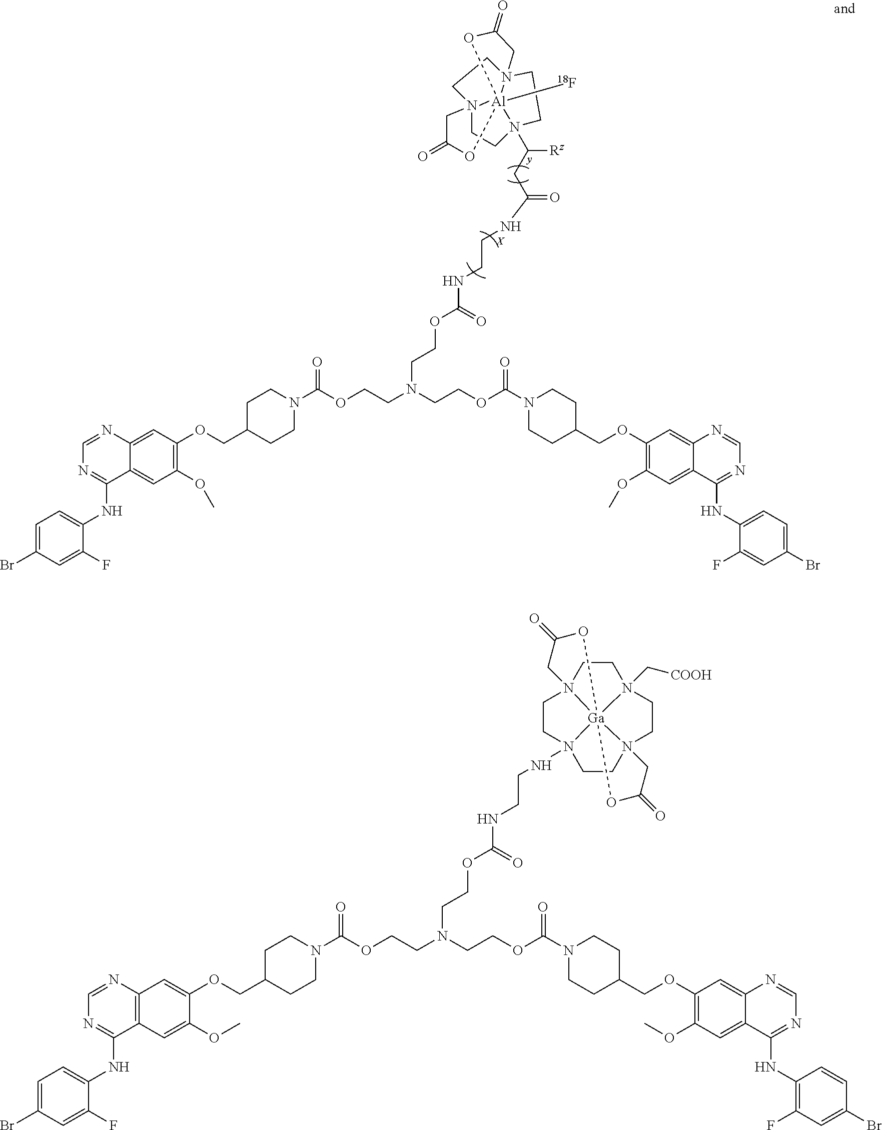

or a pharmaceutically acceptable salt thereof;
wherein:
x is 1 or 2;
y is 0 or 1; and
R^{Z} is H or COOH.

Embodiment 11. The method of any one of embodiments 1-10, wherein the disease associated with pulmonary vascular remodeling is pulmonary hypertension.

Embodiment 12. The method of embodiment 11, wherein the pulmonary hypertension is selected from: pulmonary arterial hypertension (WHO Group I); pulmonary veno-occlusive disease or pulmonary capillary hemangiomatosis (WHO Group I′); persistent pulmonary hypertension of the newborn (WHO Group I″); pulmonary hypertension secondary to left heart disease (WHO Group II); pulmonary hypertension due to lung disease or chronic hypoxia (WHO Group III); chronic arterial obstruction (WHO Group IV); and pulmonary hypertension with unclear or multifactorial mechanisms (WHO Group V).

Embodiment 13. The method of embodiment 12, wherein the pulmonary hypertension is pulmonary arterial hypertension (PAH).

Embodiment 14. The method of any one of embodiments 1-13, wherein the subject is a human.

Embodiment 15. A method of treating a disease associated with pulmonary vascular remodeling in a subject, the method comprising:
(a) administering to the subject a compound of Formula I or Formula II

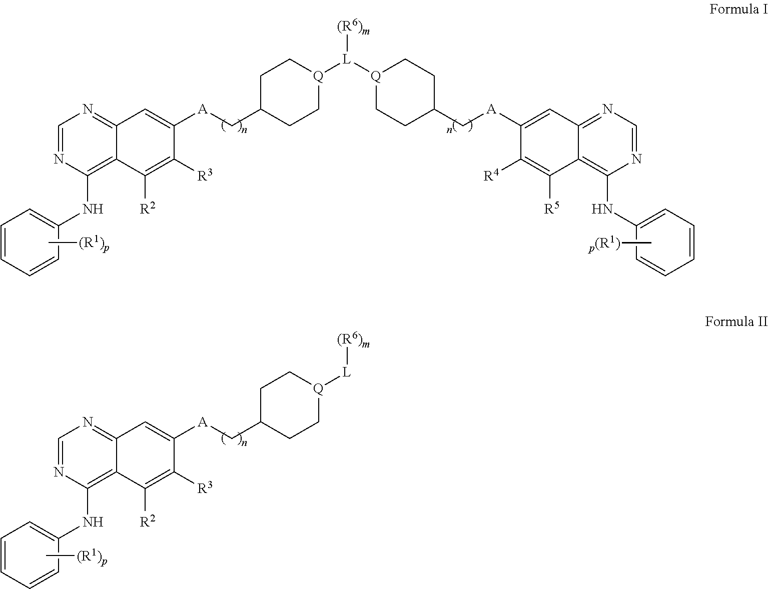

or a pharmaceutically acceptable salt thereof; and
(b) imaging the compound in lungs of the subject using positron emission tomography (PET) to detect uptake of the compound in a pulmonary vasculature of the lungs;
if retention of the compound is detected in the pulmonary vasculature in (b), then (c) administering to the subject a therapy for the disease associated with pulmonary vascular remodeling;
wherein:
R¹ is independently selected at each occurrence from hydroxyl, halogen, C₁₋₃ alkyl, C₁₋₃ alkoxyl, C₁₋₃ alkanoyloxyl, trifluoromethyl, cyano, amino, and nitro;
R², R³, R⁴, and R⁵ are independently selected from hydrogen, hydroxyl, halogen, nitro, trifluoromethyl, cyano, C₁₋₃ alkyl, C₁₋₃ alkoxyl, C₁₋₃ alkylthio, and —NR⁷R⁸,
R⁷ and R⁸ are independently at each occurrence from hydrogen and C₁₋₃ alkyl;
A is —O—, —CH₂—, —S—, —SO—, —SO₂—, —NR⁷CO—, —CONR⁷—, —SO₂NR⁷—, —NR⁷SO₂—, or —NR⁷—;
Q is N or CH;
L is a linker;
R⁶ contains a positron emission tomography (PET) detectable moiety;
n is an integer from 1 to 5;
m is an integer from 1 to 5; and
p is an integer from 1 to 4.

Embodiment 16. The method of embodiment 15, wherein the PET detectable moiety is an ¹⁸F labeled moiety or ⁶⁸Ga labeled moiety.

Embodiment 17. The method of any one of embodiments 15 or 16, wherein the PET detectable moiety is selected from:

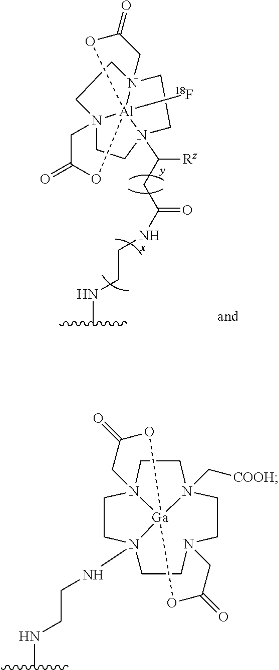

wherein:
x is 1 or 2;
y is 0 or 1; and
R^{Z} is H or COOH.

Embodiment 18. The method of any one of embodiments 15-17, wherein the compound is of Formula III

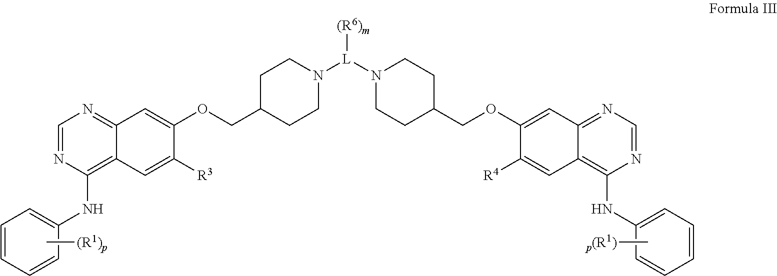

wherein:
R¹ is hydroxy, halogen, C₁₋₃ alkyl, C₁₋₃ alkoxyl, C₁₋₃ alkanoyloxyl, or trifluoromethyl; and
R³ and R⁴ are independently hydrogen, hydroxyl, halogen, C₁₋₃ alkyl, C₁₋₃ alkoxyl, or C₁₋₃ alkylthio.

Embodiment 19. The method of any one of embodiments 15-18, wherein the linker is —(CO—R¹⁴)₃N, —(R¹⁴)₃N, —(SO₂R¹⁴)₃N, —(SOR¹⁴)₃N, —(OR¹⁴)₃N, —(O—CO—R¹⁴)₃N, —(CO—O—R¹⁴)₃N, —(CO—R¹⁴)₃CH, —(R¹⁴)₃CH, —(SO₂R¹⁴)₃CH, —(SOR¹⁴)₃CH, —(O—CO—R¹⁴)₃CH, or —(OR¹⁴)₃CH.

Embodiment 20. The method of embodiment 19, wherein the linker is —(CO—O—R¹⁴)₃N or —(CO—O—R¹⁴)₃CH, wherein R¹⁴ is a C₂₋₄ alkyl.

Embodiment 21. The method of embodiment 19 or embodiment 20, wherein the linker is

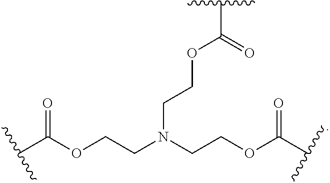 [SMILES: *C(=O)OCCN(CCOC(*)=O)CCOC(*)=O]

Embodiment 22. The method of any one of embodiments 15-21, wherein n is 1.

Embodiment 23. The method of any one of embodiments 15-22, wherein the compound is of Formula IV:

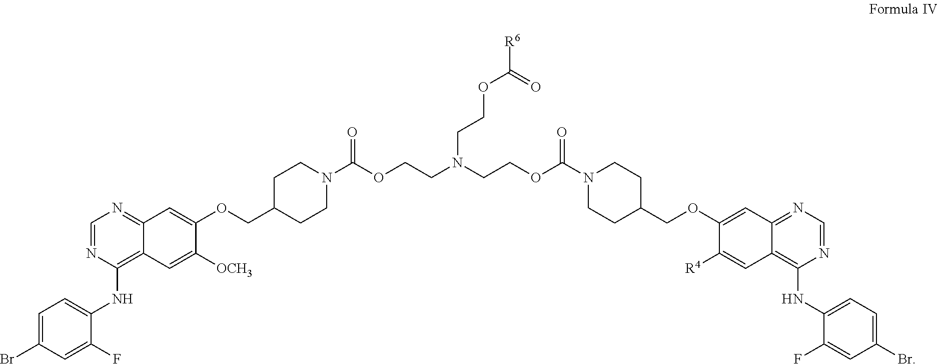

Embodiment 24. The method of any one of embodiments 15-23, wherein the compound is selected from:

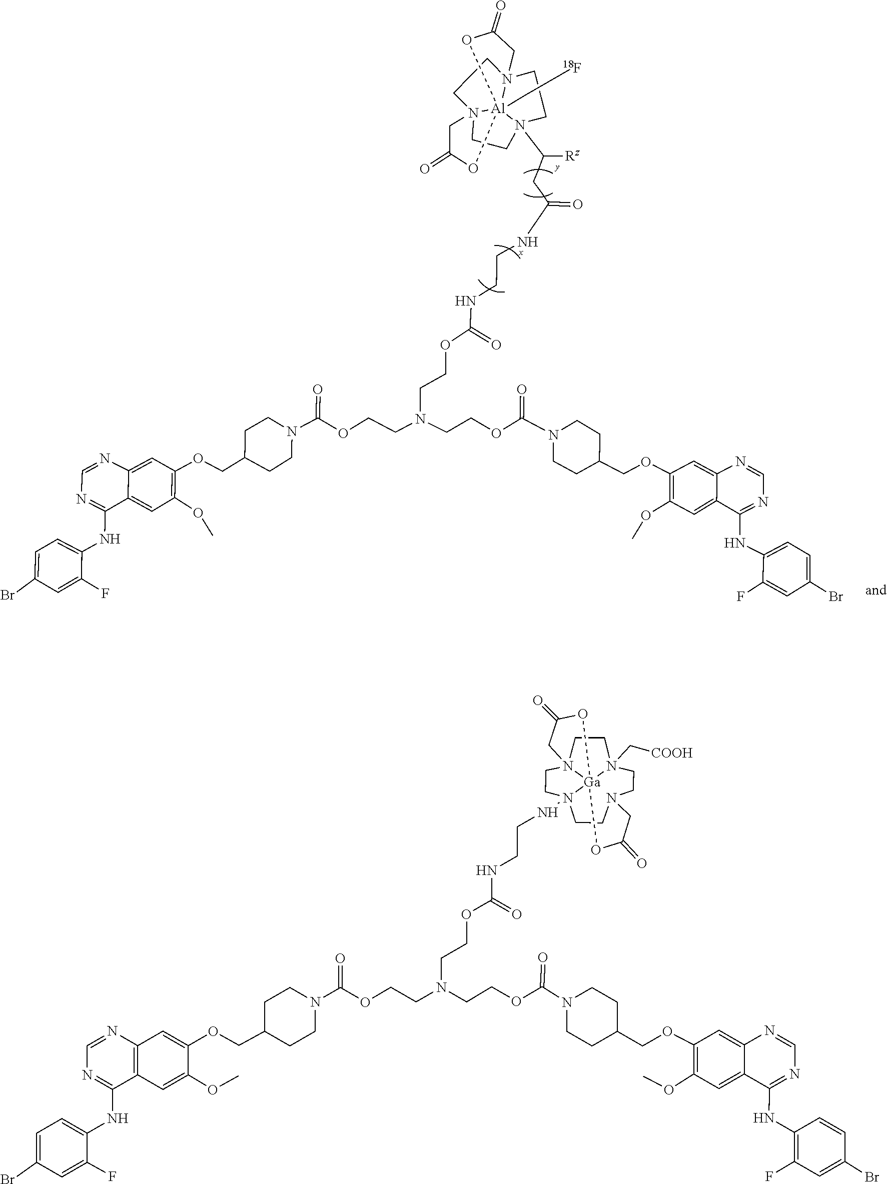

or a pharmaceutically acceptable salt thereof;
wherein:
x is 1 or 2;
y is 0 or 1; and
R^{Z} is H or COOH.

Embodiment 25. The method of any one of embodiments 15-24, wherein the disease associated with pulmonary vascular remodeling is pulmonary hypertension.

Embodiment 26. The method of embodiment 25, wherein the pulmonary hypertension is selected from: pulmonary arterial hypertension (WHO Group I); pulmonary veno-occlusive disease or pulmonary capillary hemangiomatosis (WHO Group I′); persistent pulmonary hypertension of the newborn (WHO Group I″); pulmonary hypertension secondary to left heart disease (WHO Group II); pulmonary hypertension due to lung disease or chronic hypoxia (WHO Group III); chronic arterial obstruction (WHO Group IV); and pulmonary hypertension with unclear or multifactorial mechanisms (WHO Group V).

Embodiment 27. The method of embodiment 26, wherein the pulmonary hypertension is pulmonary arterial hypertension (PAH).

Embodiment 28. The method of any one of embodiments 15-27, wherein the subject is a human.

Embodiment 29. The method of any one of embodiments 15-28, wherein the therapy comprises surgery.

Embodiment 30. The method of any one of embodiments 15-29, wherein the therapy comprises one or more additional therapeutic agent.

Embodiment 31. The method of embodiment 30, wherein the one or more additional therapeutic agents are selected from a vasodilator, a guanylate cyclase activator, an endothelin receptor antagonist, a phosphodiesterase type 5 inhibitor, a calcium channel blocker, an anticoagulant, digoxin, or a diuretic.

Embodiment 32. A method for imaging a pulmonary vasculature in a subject, the method comprising:
(a) administering to the subject a compound of Formula I or Formula II

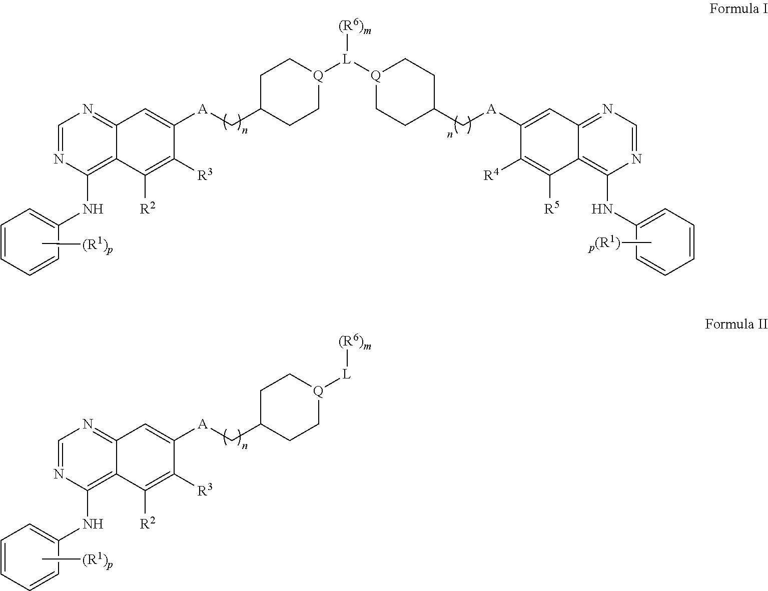

or a pharmaceutically acceptable salt thereof; and
(b) detecting a positron emission tomography (PET) signal in the pulmonary vasculature of the subject;
wherein:
R¹ is independently selected at each occurrence from hydroxyl, halogen, C₁₋₃ alkyl, C₁₋₃ alkoxyl, C₁₋₃ alkanoyloxyl, trifluoromethyl, cyano, amino, and nitro;
R², R³, R⁴, and R⁵ are independently selected from hydrogen, hydroxyl, halogen, nitro, trifluoromethyl, cyano, C₁₋₃ alkyl, C₁₋₃ alkoxyl, C₁₋₃ alkylthio, and —NR⁷R⁸,
R⁷ and R⁸ are independently at each occurrence from hydrogen and C₁₋₃ alkyl; A is —O—, —CH₂—, —S—, —SO—, —SO₂—, —NR⁷CO—, —CONR⁷—, —SO₂NR⁷—, —NR⁷SO₂—, or —NR⁷—;
Q is N or CH;
L is a linker;
R⁶ contains a positron emission tomography (PET) detectable moiety;
n is an integer from 1 to 5;
m is an integer from 1 to 5; and
p is an integer from 1 to 4.

Embodiment 33. The method of embodiment 32, wherein the PET detectable moiety is an ¹⁸F labeled moiety or ⁶⁸Ga moiety.

Embodiment 34. The method of any one of embodiments 32 or 33, wherein the PET detectable moiety is selected from:

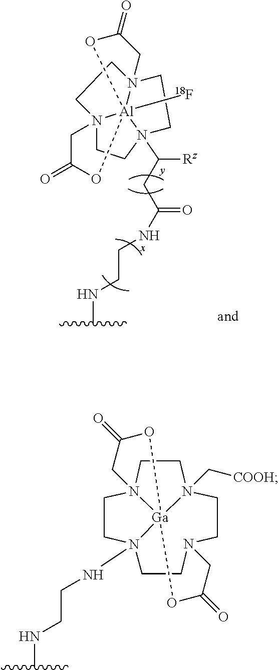

wherein:
x is 1 or 2;
y is 0 or 1; and
R^{Z} is H or COOH.

Embodiment 35. The method of any one of embodiments 32-34, wherein the compound is of Formula III

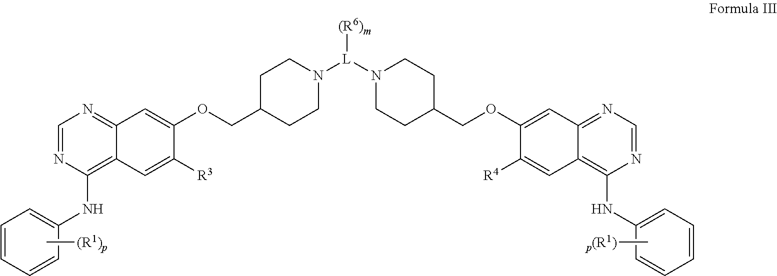

wherein:
R¹ is hydroxy, halogen, C₁₋₃ alkyl, C₁₋₃ alkoxyl, C₁₋₃ alkanoyloxyl, or trifluoromethyl; and
R³ and R⁴ are independently hydrogen, hydroxyl, halogen, C₁₋₃ alkyl, C₁₋₃ alkoxyl, or C₁₋₃ alkylthio.

Embodiment 36. The method of any one of embodiments 32-35, wherein the linker is —(CO—R¹⁴)₃N, —(R¹⁴)₃N, —(SO₂R¹⁴)₃N, —(SOR¹⁴)₃N, —(OR¹⁴)₃N, —(O—CO—R¹⁴)₃N, —(CO—O—R¹⁴)₃N, —(CO—R¹⁴)₃CH, —(R¹⁴)₃CH, —(SO₂R¹⁴)₃CH, —(SOR¹⁴)₃CH, —(O—CO—R¹⁴)₃CH, or —(OR¹⁴)₃CH.

Embodiment 37. The method of embodiment 36, wherein the linker is —(CO—O—R¹⁴)₃N or —(CO—O—R¹⁴)₃CH, wherein R¹⁴ is a C₂₋₄ alkyl.

Embodiment 38. The method of embodiment 36 or embodiment 37, wherein the linker is

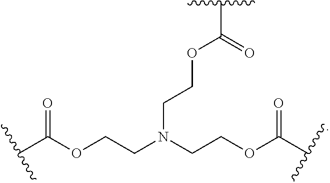 [SMILES: *C(=O)OCCN(CCOC(*)=O)CCOC(*)=O]

Embodiment 39. The method of any one of embodiments 32-38, wherein n is 1.

Embodiment 40. The method of any one of embodiments 32-39, wherein the compound is of Formula IV:

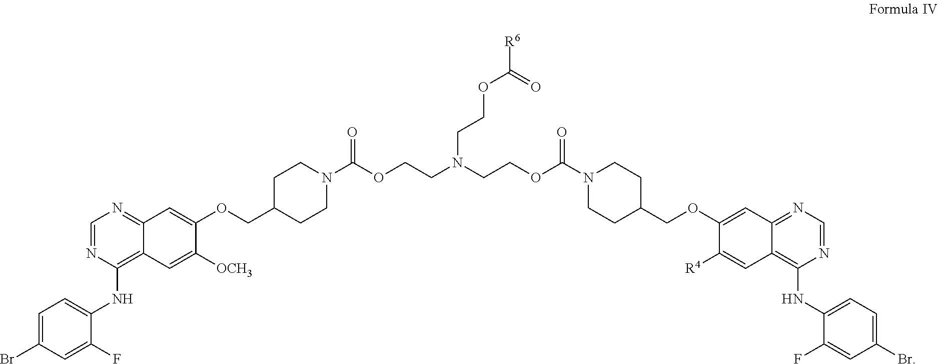

Embodiment 41. The method of any one of embodiments 32-40, wherein the compound is selected from:

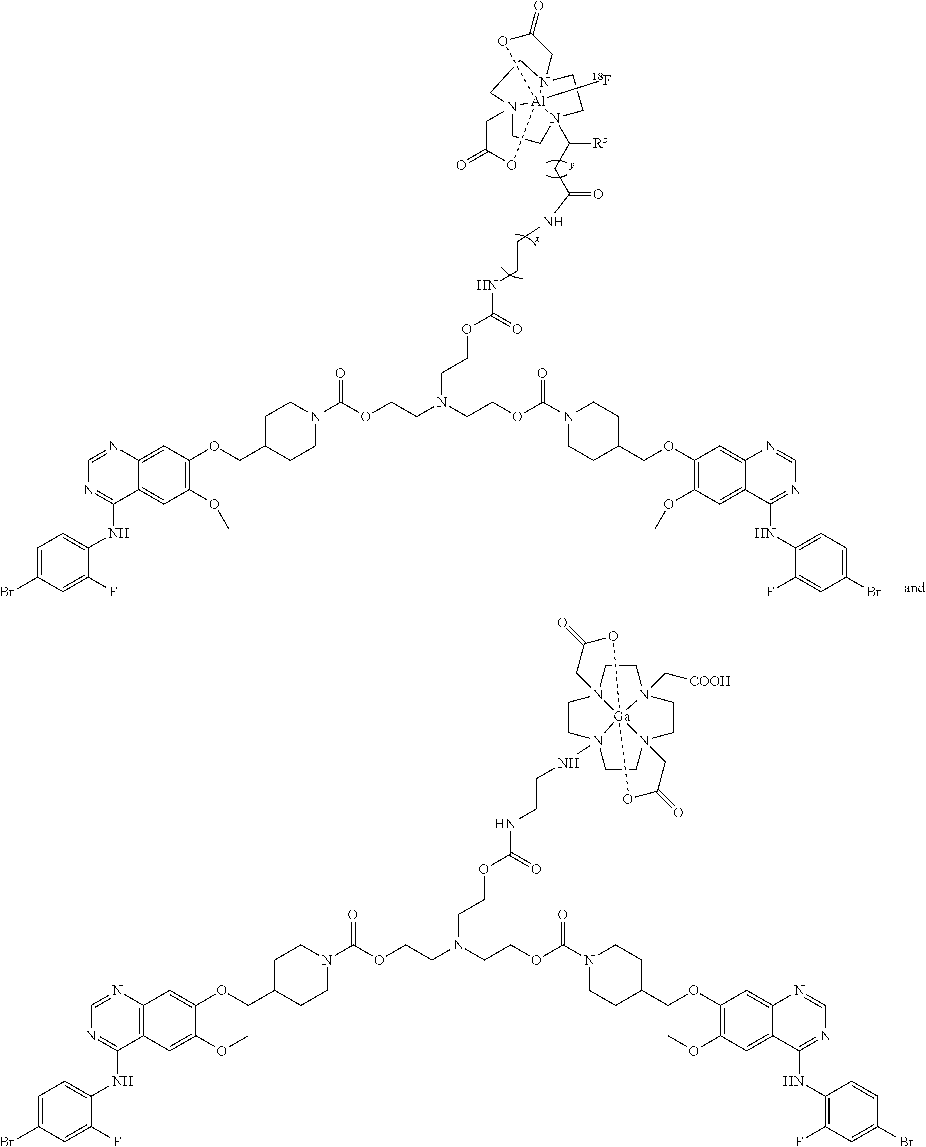

or a pharmaceutically acceptable salt thereof;
wherein:
x is 1 or 2;
y is 0 or 1; and
R^{Z} is H or COOH.

Embodiment 42. The method of any one of embodiments 32-41, wherein the subject is a human.

Embodiment 43. A compound selected from:

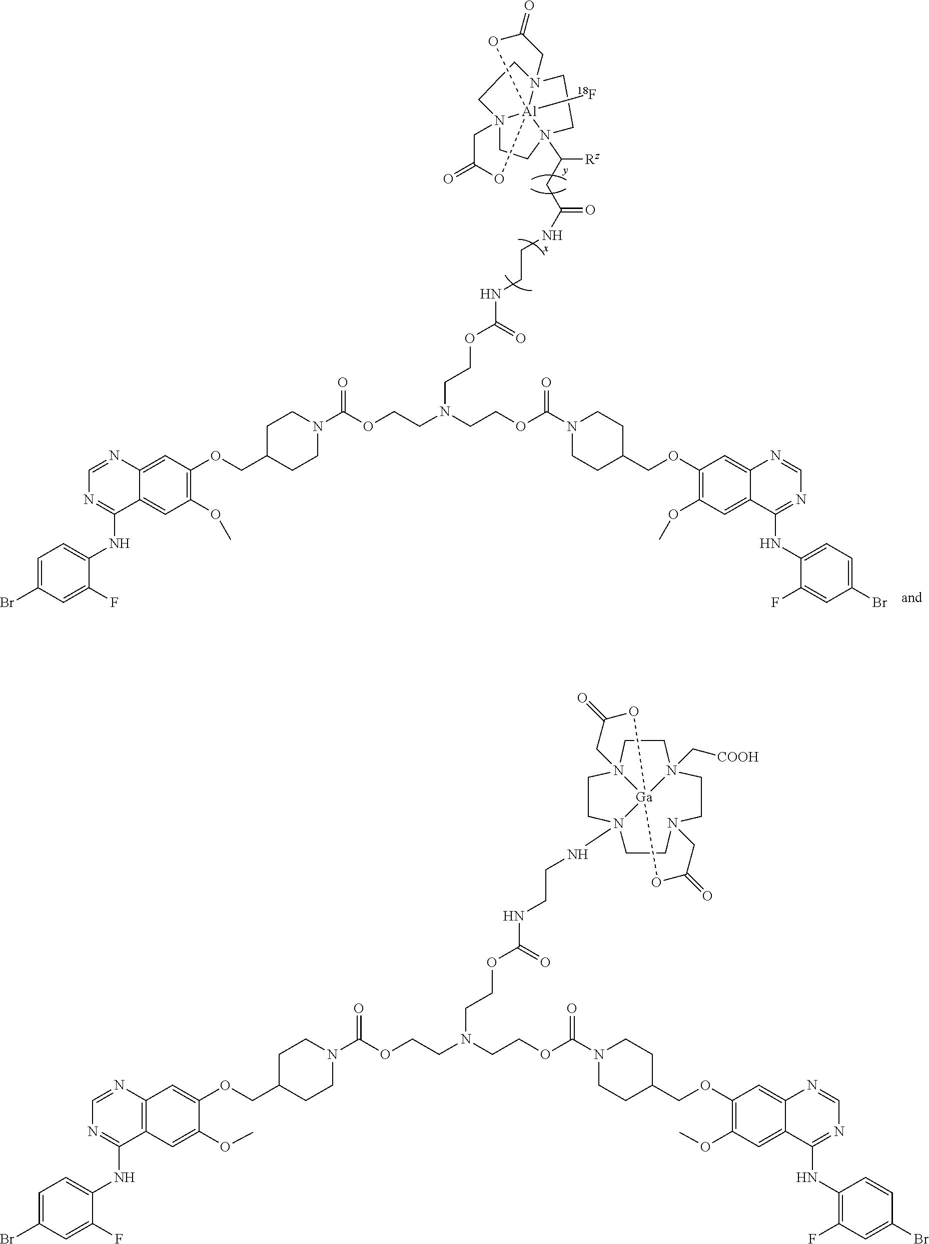

or a pharmaceutically acceptable salt thereof;
wherein:
x is 1 or 2;
y is 0 or 1; and
R^{Z} is H or COOH.

Embodiment 44. A pharmaceutical composition comprising the compound of embodiment 43, or a pharmaceutically acceptable salt thereof, and a pharmaceutically acceptable carrier or excipient.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth so as to provide those of ordinary skill in the art with a complete disclosure and description of how the compounds, compositions, devices and/or methods described and claimed herein are made and evaluated and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric.

### High Affinity VEGFr2 Radiotracer Development

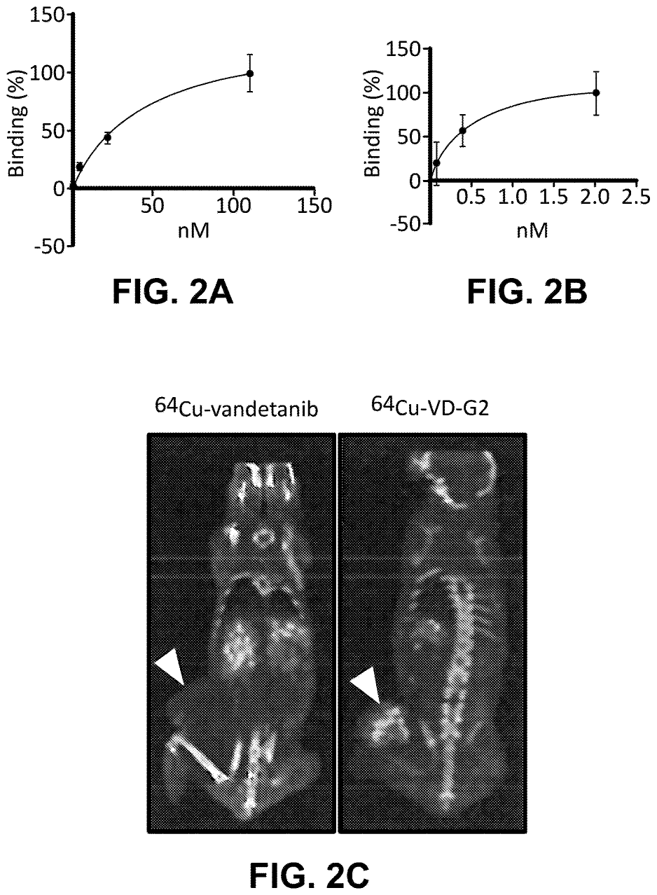

Vandetanib is an orally bioavailable anti-angiogenic quinazoline drug selective for the tyrosine kinase activity of VEGFr2 with a K_{d} value of 40 nM. Based on the structure of vandetanib, we developed a lead compound (ZD-G2) with improved binding to VEGFr2 in sub-nanomolar range (K_{d} 0.45±0.32 nM). This compound demonstrated approximately two logarithmic orders of magnitude improvement in VEGF receptor binding affinity in vitro compared to vandetanib and significantly enhanced tumor localization (FIG. 2). PET imaging using U87 glioblastoma xenograft models demonstrated 7-fold higher tumor uptake of the developed agent than that of vandetanib while maintaining pharmacokinetic and biodistribution properties well suited for PET imaging. To assess VEGFr2 specificity in vivo, we also performed blocking experiments by co-injection of nonradioactive VEGFr inhibitor with ⁶⁴Cu-ZD-G2. Tumor activity was effective reduced at all-time points in the blocking study, which indicated specific VEGFr binding in vivo.

### Cy5.5-ZD-G2 Staining Using PAH Human Lung

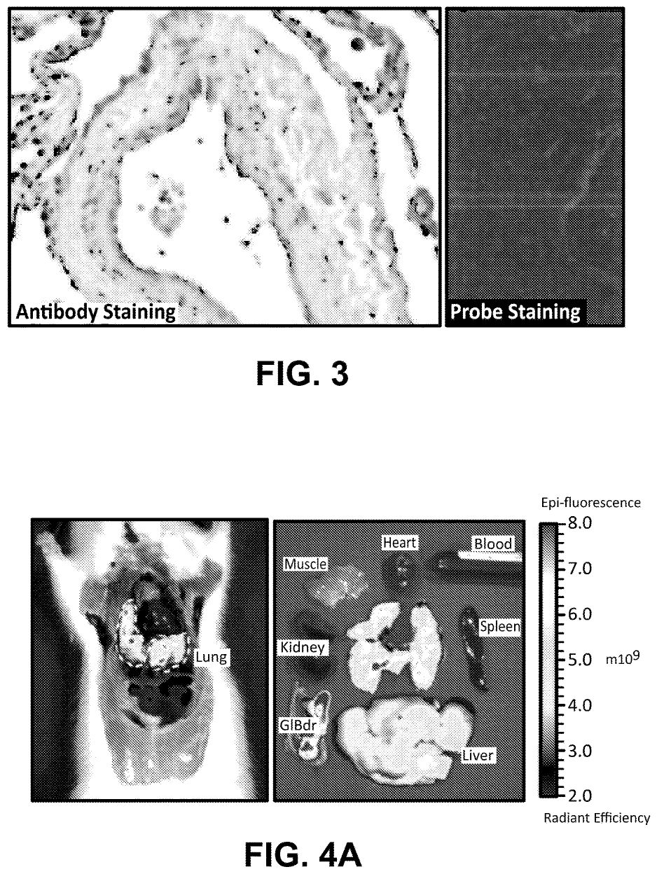

To study the binding of ZD-G2 to pulmonary arteries of PAH patients we used explanted lung tissue from lung transplant patients with PAH. We synthesized a fluorescence probe Cy5.5-ZD-G2 by conjugated fluorophore Cy5.5 to ZD-G2 for human PAH lung staining. As shown in FIG. 3, fluorescence Cy5.5-ZD-G2 staining shows an excellent correspondence to the VEGFr2 immunohistochemistry staining on the arterial endothelial cells in the PAH human lung. It gives compelling evidence that the developed ZD-G2 radiotracer shows promise as a noninvasive imaging tool for diagnosis of PAH.

### Near-Infrared (NIR) Optical Imaging Using MCT Rat Model

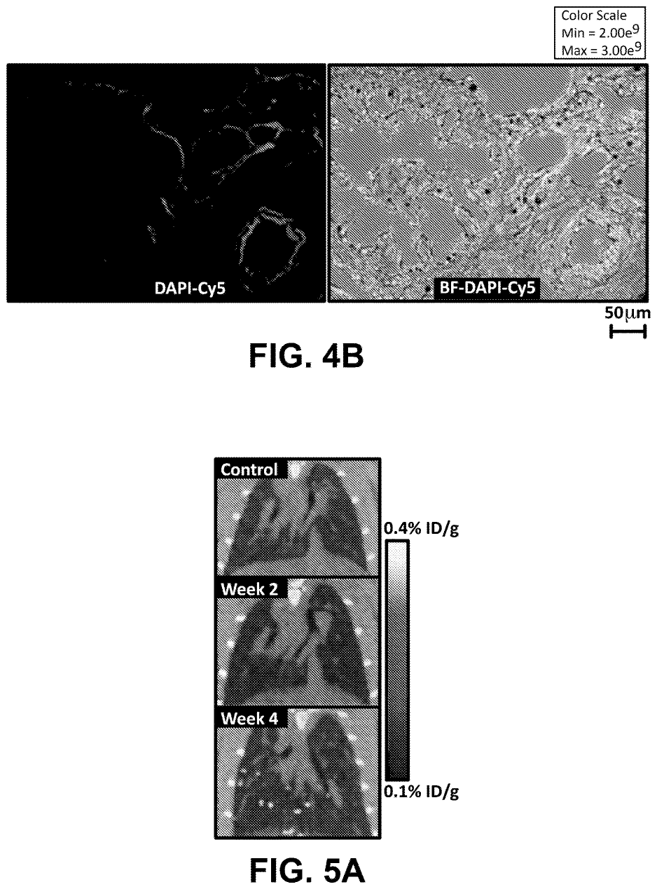

A rat model of PAH was generated using 2-month-old Sprague Dawley rats by IP injection of monocrotaline (50 mg/lg), which has been extensively studied and characterized. NIR optical imaging of Cy5.5-ZD-G2 was carried out using MCT rats 4 weeks after injection of monocrotaline. Fluorescence imaging were performed with an IVIS200 imaging system and quantified by Living Imaging software (Xenogen, Alameda, CA). Excitation and emission filters were set at 675 and 720 nm, respectively, as suggested by the system for image acquisition. Each rat was injected intravenously with 10 nmol of Cy5.5-ZD-G2 conjugate. After 30 mins, rats were sacrifice, and lungs, heart, muscles, and other major organs (i.e., liver, spleen, kidney, and intestines) were excised for ex vivo imaging acquisition. As shown in FIG. 4, the imaging results clearly shows the specific uptake of the Cy5.5-ZD-G2 probe in the PAH rat lung, while the histology sample further supports the staining of the Cy5.5-ZD-G2 on the arterioles of the PAH lung tissue. The immunohistochemistry staining also confirmed high VEGFr2 expression in the rat PAH lung.

### Positron Emission Tomography (PET) Imaging of 64Cu-ZD-G2 Using MCT Rat Model

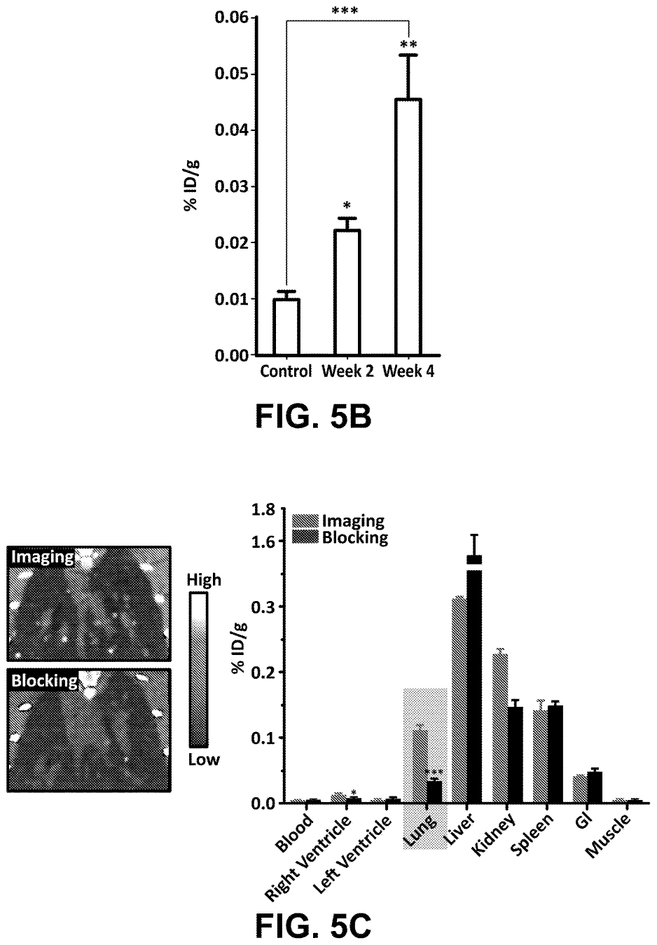

A PET imaging study was carried out with 64Cu-ZD-G2 using MCT rat model that is generated by one-time IP injected on monocrotaline (MCT, 50 mg/kg), previously described. We quantified changes in the uptake of 64Cu-ZD-G2 by in vivo PET imaging, mean pulmonary artery pressure (mPAP) and cardiac output using echocardiography, and invasive cardiac catheterization at different time points (week 2 and 4). At week 2, no lung pressure change was observed compared to control mice (no PAH). As shown in FIG. 5, lung uptake can be clearly visualized at the early stage of week 2 and increased lung uptake is observed along the PAH progression at week 4. The result in the blocking studies demonstrated the probe specificity.

### Development of 18F-ZD-G2

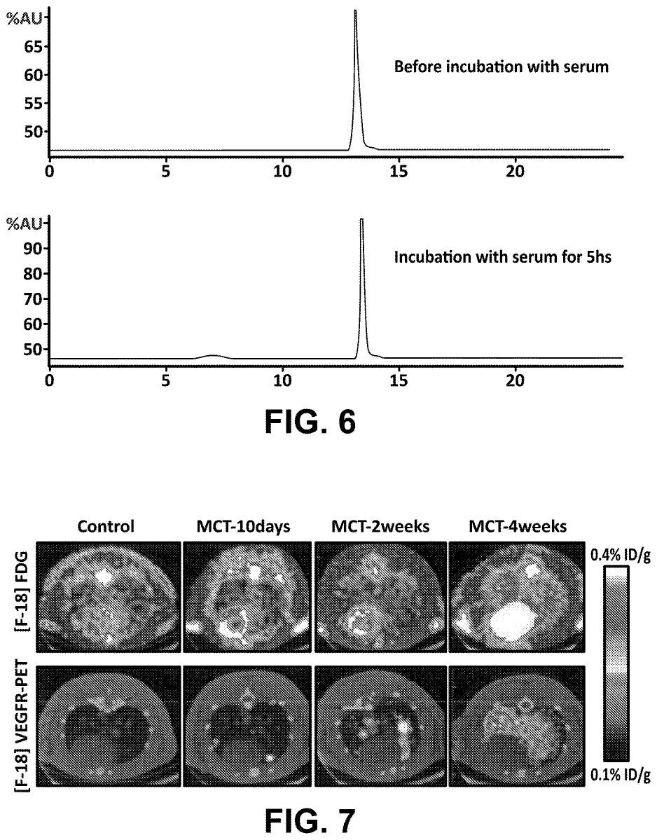

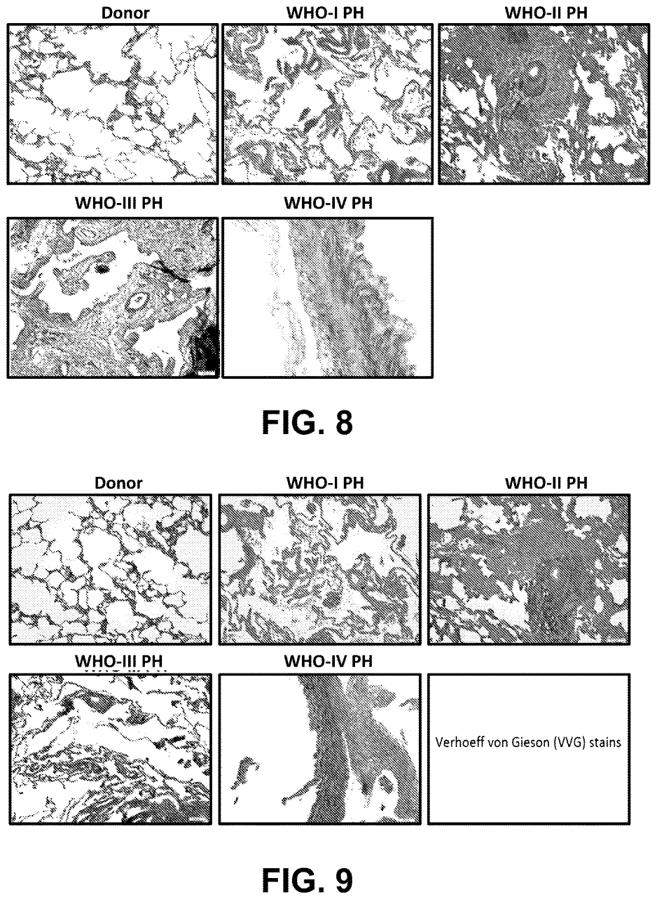

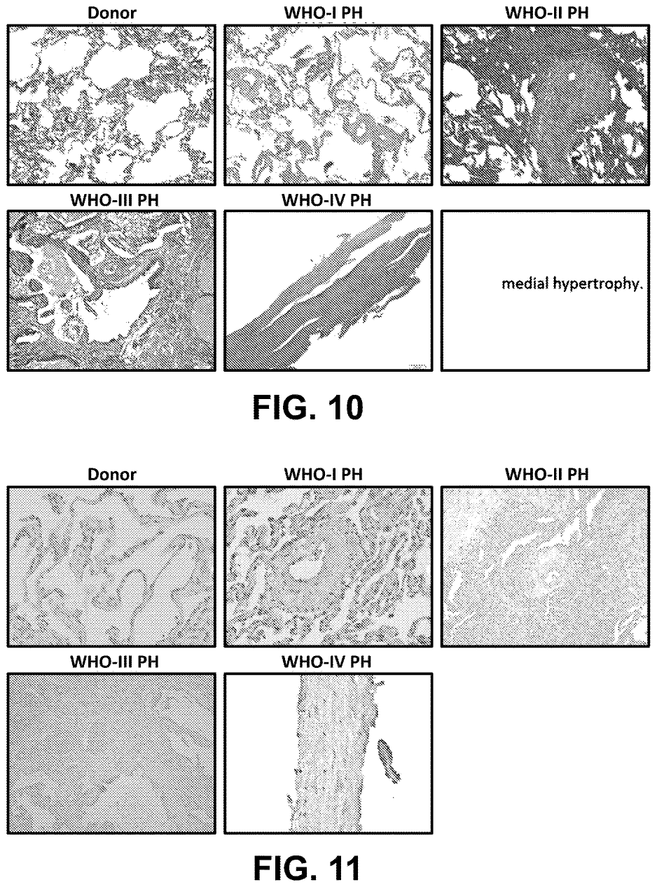

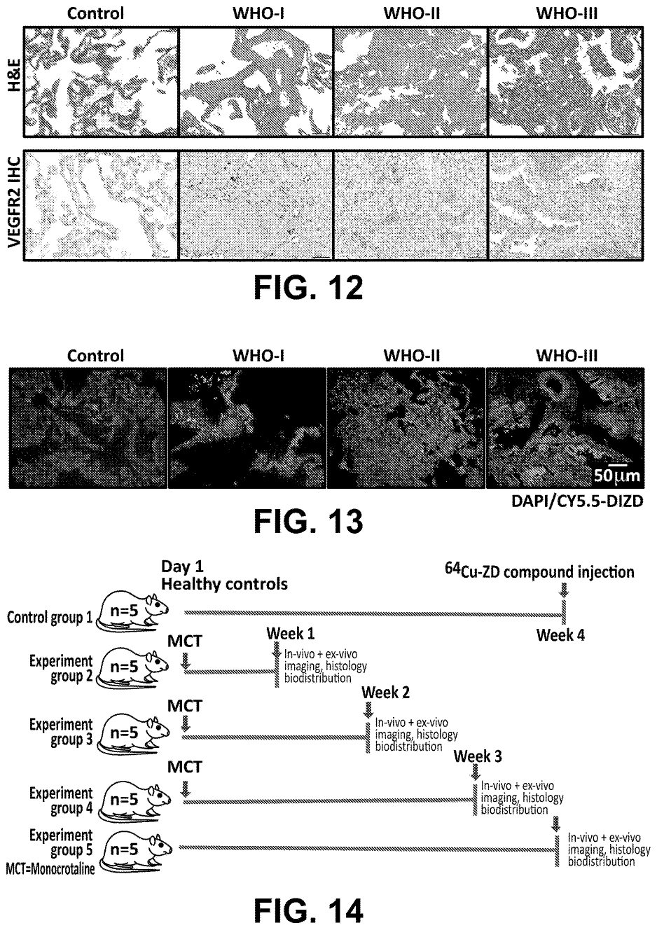

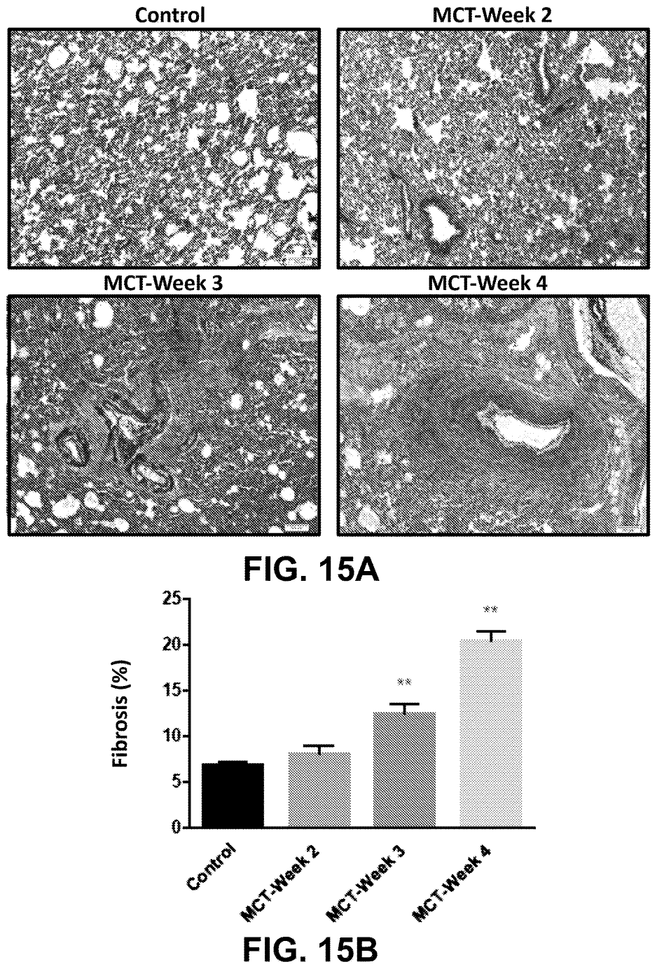

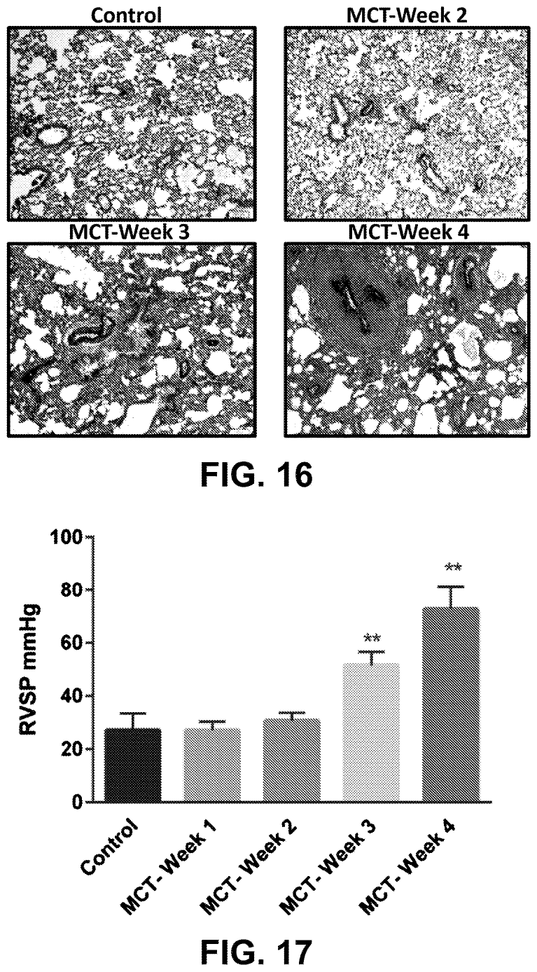

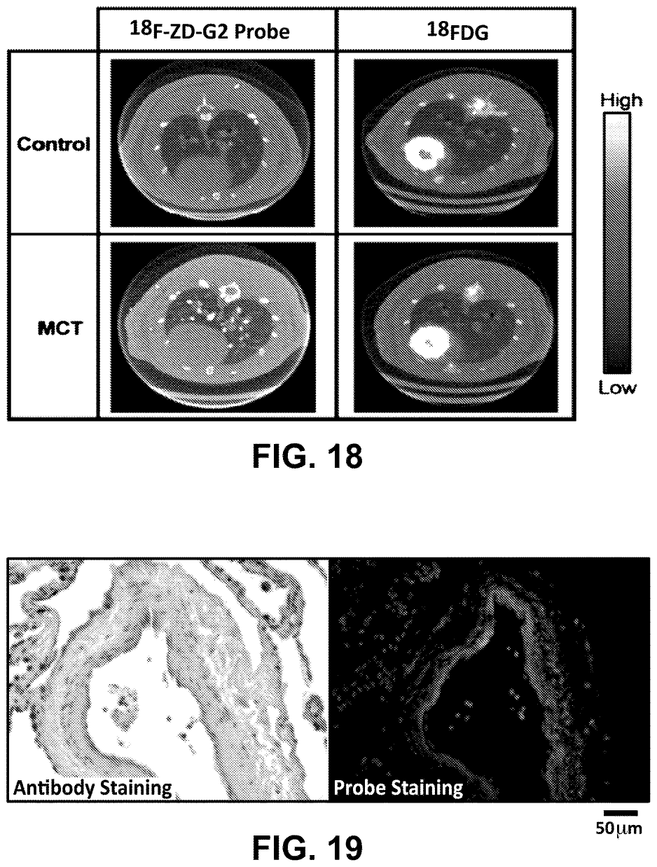

Radiolabeling compounds with positron-emitting radionuclides like ¹⁸F often involves a time-consuming, customized process. Here, we successfully radiolabeled ZD-G2 with a rapid one-step ¹⁸F labeling reaction with fluoridealuminum complex. The whole radiosynthesis was accomplished within 35 min with radiochemical purity of more than 98% (n=8). The specific activity of purified ¹⁸F-ZD-G2 was calculated as 450-500 mCi/micromole. As shown in FIG. 6, chemical stability of ¹⁸F-ZD-G2 was evaluated by radio-HPLC analysis that revealed no change in the chromatograph after 5 h incubation in mouse serum at 37 °C.

### PET Imaging of ¹⁸F-ZD-G2 Using MCT Rat Model

A PET imaging study was further performed with ¹⁸F-ZD-G2 using MCT rat model as previous described. Healthy rats were used as control. Clinically used ¹⁸FDG PET imaging was also performed on both groups of mice following the same imaging protocol for comparison. As shown in FIG. 7, increased lung uptake of ¹⁸F-ZD-G2 was visualized in MCT mice compared to the control mice, but no obvious difference showed with ¹⁸FDG PET in both mice groups at early stage of PAH (MCT-10 days and MCT-2 weeks). Beside the PAH lung uptake, there is much lowered heart uptake and more clean background in ¹⁸F-ZD-G2 PET images in comparison to ¹⁸FDG PET. As we know that ¹⁸FDG is a non-specific radiopharmaceutical for tissue metabolism, our result demonstrated that ¹⁸F-ZD-G2, which specifically targets the PAH biomarker VEGFr2, could effectively detect PAH at the early stage and have great potential for clinical translation.

### Synthetic Methods

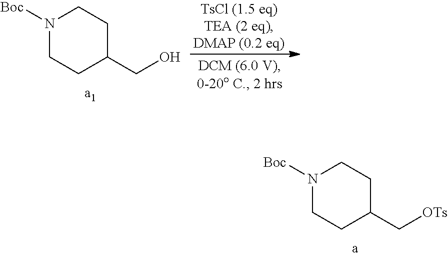 [SMILES: CC(C)(C)OC(=O)N1CCC(CO)CC1 ; Cc1ccc(S(=O)(=O)OCC2CCN(C(=O)OC(C)(C)C)CC2)cc1]

Compound a obtained as a yellow solid after work up (washed with saturated citric acid solution and 1 M NaOH, extracted with DCM two times) and purification (triturated with n-heptane:EtOAc=10:1).

| Entry | a₁ (g) | % Yield | % Purity | Yield (g) |
|---|---|---|---|---|
| 1 | 10.0 | 68.8 | 98.5 | 12.0 |
| 2 | 100 | 71.7 | 98.5 | 125 |
| 3 | 295 | 79.2 | 97.8 | 685 |
| | 295 | | | |

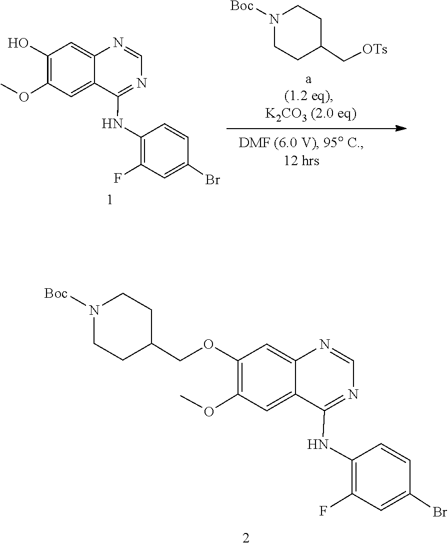 [SMILES: COc1cc2c(Nc3ccc(Br)cc3F)ncnc2cc1O ; Cc1ccc(S(=O)(=O)OCC2CCN(C(=O)OC(C)(C)C)CC2)cc1 ; COc1cc2c(Nc3ccc(Br)cc3F)ncnc2cc1OCC1CCN(C(=O)OC(C)(C)C)CC1]

Compound 2 obtained as a yellow solid after work up (pour into H₂O, filter, and concentrate the cake in vacuum) and purification (triturated with MeOH).

| Entry | 1 (g) | % Yield | % Purity | Yield (g) |
|---|---|---|---|---|
| 1 | 10.0 | 76.5 | 99.7 | 11.8 |
| 2 | 100 | 65.7 | 96.6 | 105 |
| 3 | 495 | 74.7 | 98.4 | 580 |

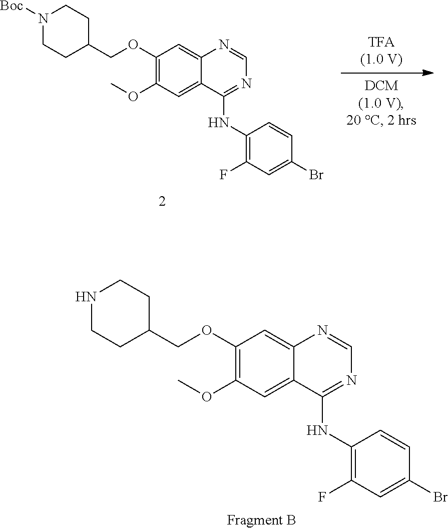 [SMILES: COc1cc2c(Nc3ccc(Br)cc3F)ncnc2cc1OCC1CCN(C(=O)OC(C)(C)C)CC1 ; COc1cc2c(Nc3ccc(Br)cc3F)ncnc2cc1OCC1CCNCC1 ; "Fragment B"]

Fragment B obtained as a yellow solid after work up (concentrate the reaction mixture in vacuum, then dissolve the crude product in MeOH, adjust to pH=9 with solid NaHCO₃, filter) and purification (triturate the filter cake with DCM).

| Entry | 2 (g) | % Yield | % Purity | Yield (g) |
|---|---|---|---|---|
| 1 | 1.80 | 87.1 | 99.2 | 1.3 |
| 2 | 10.0 | 94.1 | 99.2 | 7.8 |
| 3 | 105 | 92.9 | 99.5 | 80.2 |
| 4 | 580 | 97.7 | 99.7 | 466 |

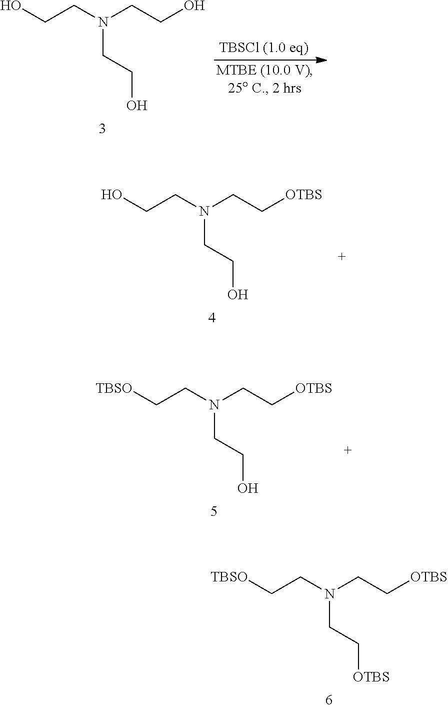 [SMILES: OCCN(CCO)CCO ; CC(C)(C)[Si](C)(C)OCCN(CCO)CCO ; CC(C)(C)[Si](C)(C)OCCN(CCO)CCO[Si](C)(C)C(C)(C)C ; CC(C)(C)[Si](C)(C)OCCN(CCO[Si](C)(C)C(C)(C)C)CCO[Si](C)(C)C(C)(C)C]

Compound 4 and Compound 5 obtained after work up (pour into water, separate the organic layer, and concentrate under vacuum) and purification (by column chromatography).

| Entry | 3 (g) | Yield 4 (g) | Yield 5 (g) |
|---|---|---|---|
| 1 | 2 | 300 mg | 580 mg |
| 2 | 25 | 3.5 | 5.5 |
| 3 | 100 | | 22 |
| 4 | 1 kg | | 225 |

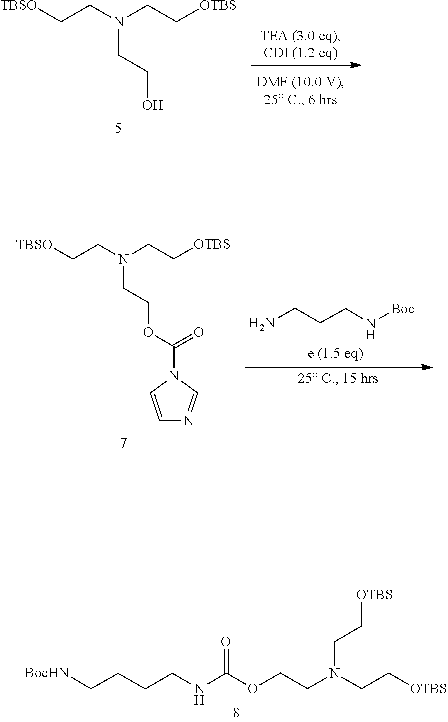 [SMILES: CC(C)(C)[Si](C)(C)OCCN(CCO)CCO[Si](C)(C)C(C)(C)C ; CC(C)(C)[Si](C)(C)OCCN(CCOC(=O)n1ccnc1)CCO[Si](C)(C)C(C)(C)C ; CC(C)(C)OC(=O)NCCCN ; CC(C)(C)OC(=O)NCCCCNC(=O)OCCN(CCO[Si](C)(C)C(C)(C)C)CCO[Si](C)(C)C(C)(C)C]

Compound 8 obtained as a yellow oil after work up (pour into water, separate the organic layer, and concentrate under vacuum).

| Entry | 5 (g) | Yield 8 (g) | % Purity |
|---|---|---|---|
| 1 | 500 mg | 620 mg | 85 (HNMR) |
| 2 | 5.48 | 7.8 | 90.9 (LCMS) |
| 3 | 22 | 32.5 | 89.1 (LCMS) |
| 4 | 225 | 320 | |

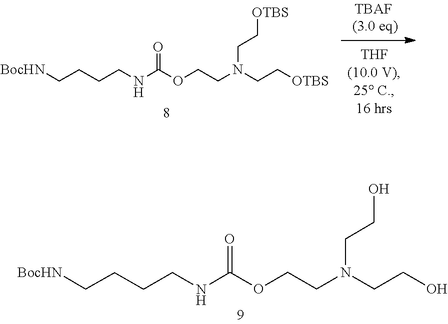 [SMILES: CC(C)(C)OC(=O)NCCCCNC(=O)OCCN(CCO[Si](C)(C)C(C)(C)C)CCO[Si](C)(C)C(C)(C)C ; CC(C)(C)OC(=O)NCCCCNC(=O)OCCN(CCO)CCO]

Compound 9 was obtained as a yellow oil after work up (pour into water, separate the organic layer, and concentrated under vacuum).

| Entry | 8 (g) | Yield 9 (g) | % Purity |
|---|---|---|---|
| 1 | 16 mg | | |
| 2 | 620 mg | 320 mg | |
| 3 | 7.8 | 4.1 | 80 (HNMR) |
| 4 | 32.5 | 17.5 | 85 (HNMR) |
| 5 | 320 | 169 | 83 (HNMR) |

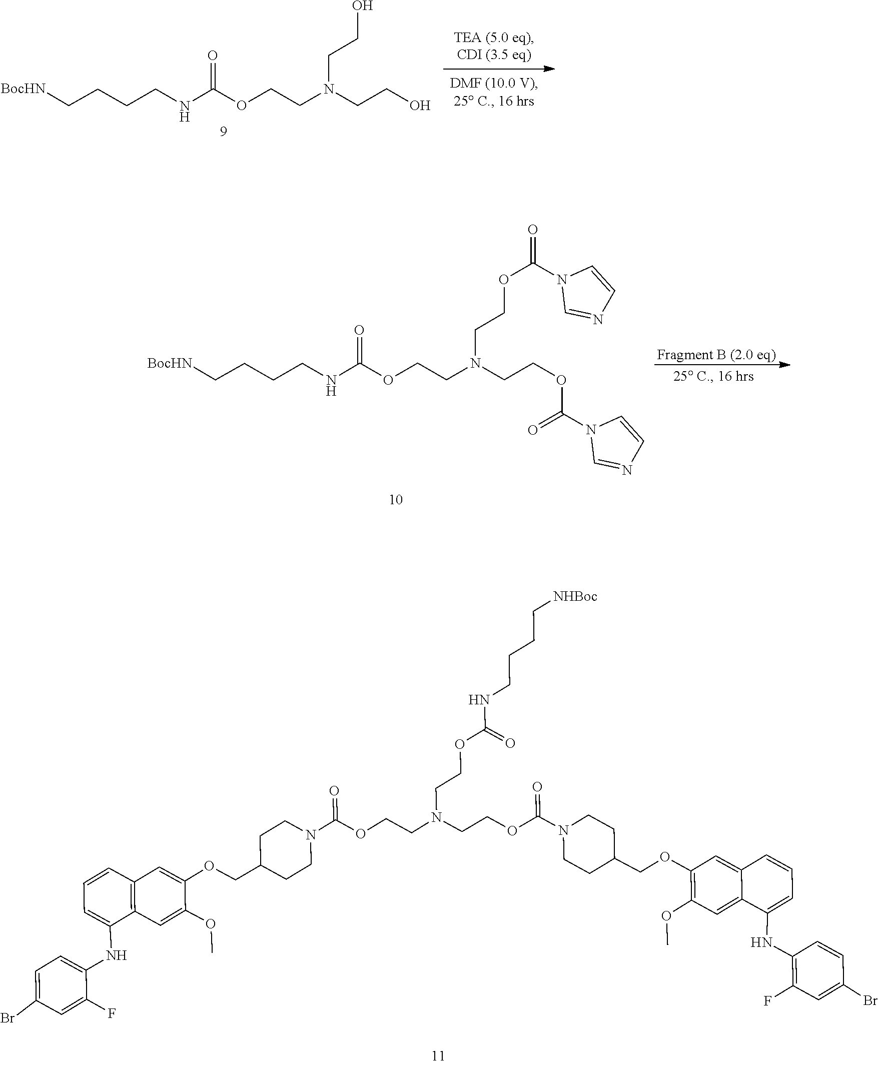 [SMILES: CC(C)(C)OC(=O)NCCCCNC(=O)OCCN(CCO)CCO ; CC(C)(C)OC(=O)NCCCCNC(=O)OCCN(CCOC(=O)n1ccnc1)CCOC(=O)n1ccnc1 ; COc1cc2c(Nc3ccc(Br)cc3F)cccc2cc1OCC1CCN(C(=O)OCCN(CCOC(=O)NCCCCNC(=O)OC(C)(C)C)CCOC(=O)N2CCC(COc3cc4cccc(Nc5ccc(Br)cc5F)c4cc3OC)CC2)CC1]

Compound 11 obtained as a brown solid after work up (pour into water, separate the organic layer, and concentrate under vacuum).

| Entry | 9 (g) | Yield 11 (g) | % Purity |
|---|---|---|---|
| 1 | 4.42 | 4.3 | |
| 2 | 17.5 | 22.0 | 93 (LCMS) |
| 3 | 169 | 175 | 91 (LCMS) |

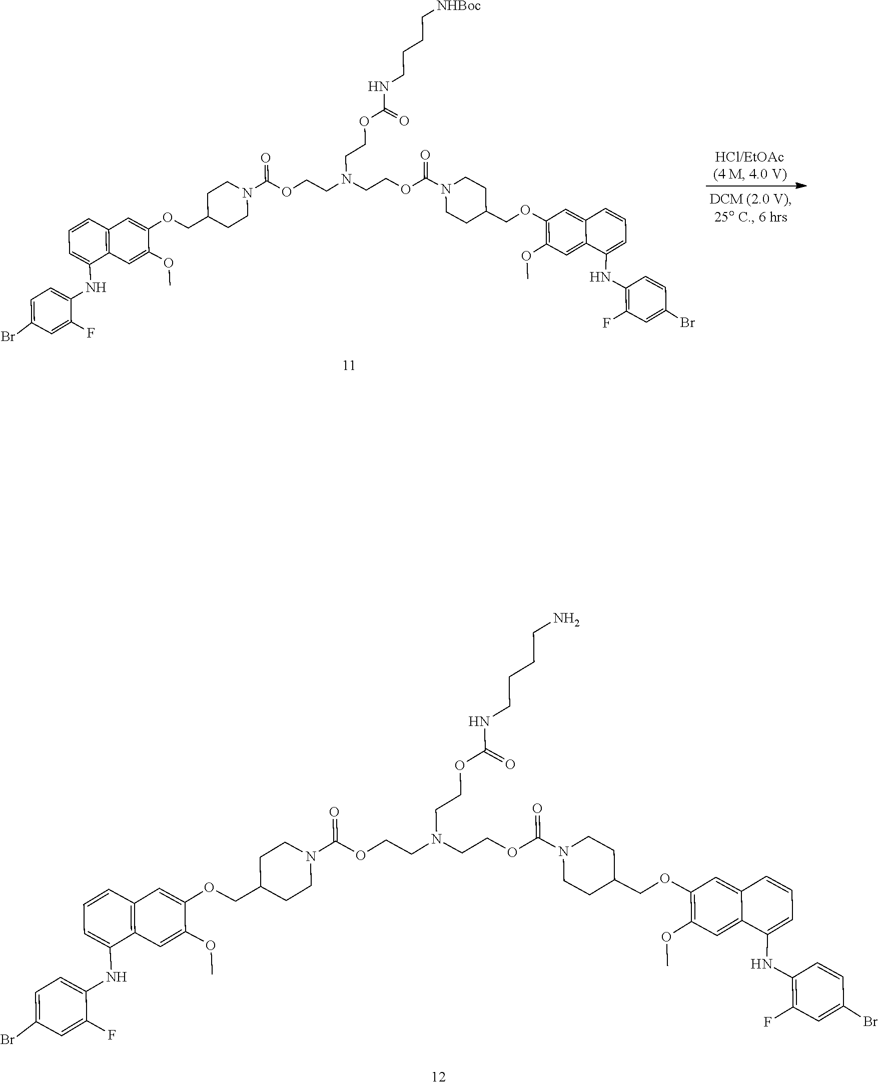 [SMILES: COc1cc2c(Nc3ccc(Br)cc3F)cccc2cc1OCC1CCN(C(=O)OCCN(CCOC(=O)NCCCCNC(=O)OC(C)(C)C)CCOC(=O)N2CCC(COc3cc4cccc(Nc5ccc(Br)cc5F)c4cc3OC)CC2)CC1 ; COc1cc2c(Nc3ccc(Br)cc3F)cccc2cc1OCC1CCN(C(=O)OCCN(CCOC(=O)NCCCCN)CCOC(=O)N2CCC(COc3cc4cccc(Nc5ccc(Br)cc5F)c4cc3OC)CC2)CC1]

Compound 12 obtained as an off-white solid after work up (filter, dissolve the filter cake into water, adjust the pH of the mixture to 8-9, extract with DCM, separate the organic layer, and concentrate under vacuum).

| Entry | 11 (g) | Yield 12 (g) | % Purity |
|---|---|---|---|
| 1 | 4.3 | 1.76 | 93 (LCMS) |
| 2 | 22 | 12.6 | 97 (LCMS) |
| 3 | 175 | 88.5 | 90 (LCMS) |

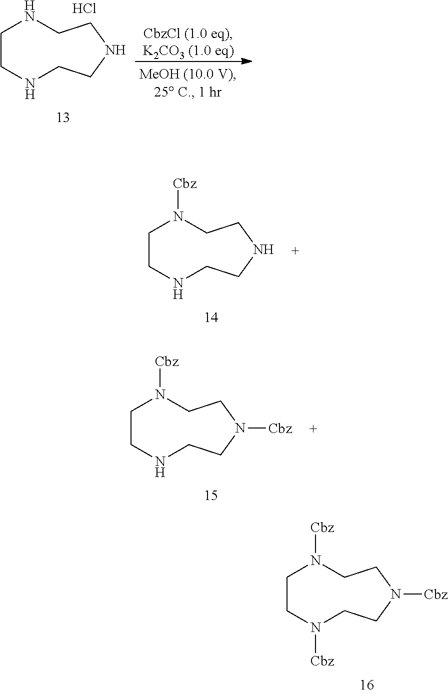 [SMILES: Cl ; C1CNCCNCCN1 ; O=C(OCc1ccccc1)N1CCNCCNCC1 ; O=C(OCc1ccccc1)N1CCNCCN(C(=O)OCc2ccccc2)CC1 ; O=C(OCc1ccccc1)N1CCN(C(=O)OCc2ccccc2)CCN(C(=O)OCc2ccccc2)CC1]

Compound 13 obtained as a yellow oil after work up (pour into water, separate the organic layer, and concentrate under vacuum) and purification (by column chromatography).

| Entry | 13 (g) | Yield 15 (g) | % Purity |
|---|---|---|---|
| 1 | 2 | 700 mg | 90.6 (LCMS) |
| 2 | 20 | 11.2 | 93.5 (LCMS) |
| 3 | 100 | 36.5 (39.0 16) | 82 (LCMS) |
| 4 | 478 | 135 (168 16) | 85.7 (LCMS) |

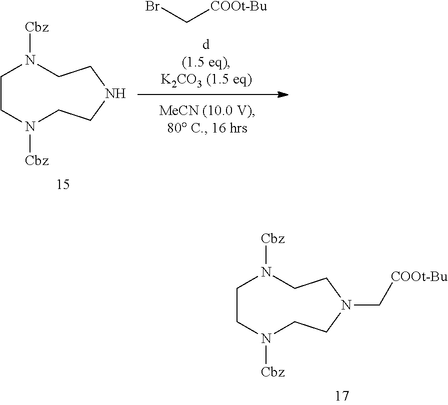

Compound 17 obtained as a yellow oil after work up (pour into water, separate the organic layer, and concentrate under vacuum) and purification (by column chromatography).

| Entry | 15 (g) | Yield 17 (g) | % Purity |
|---|---|---|---|
| 1 | 11 | 7.2 | 89.6 (LCMS) |
| 2 | 36.5 | 23.6 | |
| 3 | 135 | 90.2 | |

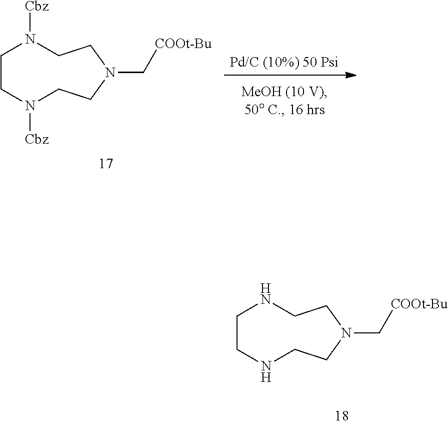

Compound 18 obtained as a yellow oil after work up (filter and concentrate the filtrate under vacuum).

| Entry | 17 (g) | Yield 18 (g) |
|---|---|---|
| 1 | 1 | 360 mg |
| 2 | 6.2 | 2.7 |
| 3 | 23.6 | 9.8 |
| 4 | 90.2 | 38 |

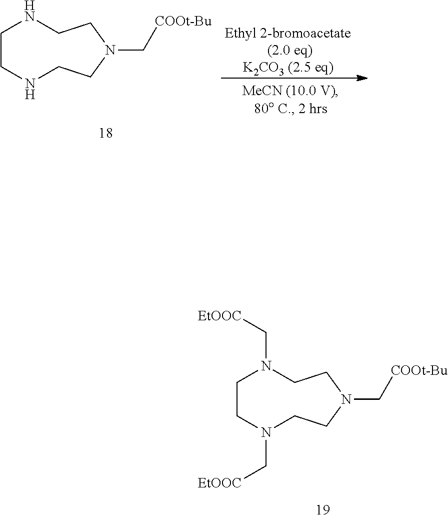

Three separate sets of conditions were evaluated. The above conditions were found to be the best compared to the alternatives tested (Ethyl 2-oxoacetate (2 eq), NaBH(OAC)₃ (2.5 eq), DCM (20.0 V), 20 °C, 6 hrs; or Ethyl 2-oxoacetate (2 eq), NaBH₃CN (2.5 eq), MeOH (20.0 V), 20 °C, 6 hrs). Compound 19 was obtained as a yellow oil after work up (pour into water, separate the organic layer, and concentrate under vacuum).

| Entry | 18 (g) | Yield 19 (g) |
|---|---|---|
| 1 | 260 mg | 82 mg |
| 2 | 2.7 | 3.2 |
| 3 | 9.8 | 10.6 |
| 4 | 38 | 46.0 |

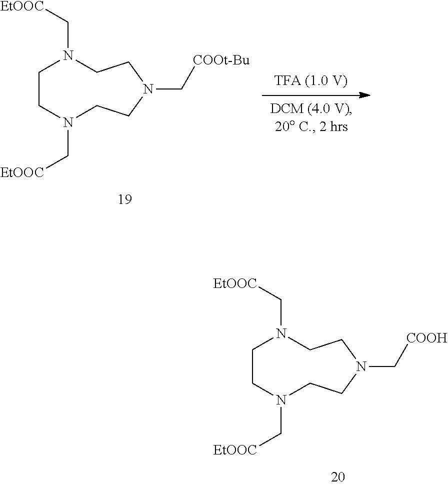

Compound 20 obtained as a yellow after work up (pour into water, separate the organic layer, and concentrate under vacuum) and purification (by preparatory HPLC).

| Entry | 19 (g) | Yield 20 (g) |
|---|---|---|
| 1 | 3.28 | 1.55 |
| 2 | 10.6 | 3.4 |
| 3 | 46 | 28.6 |

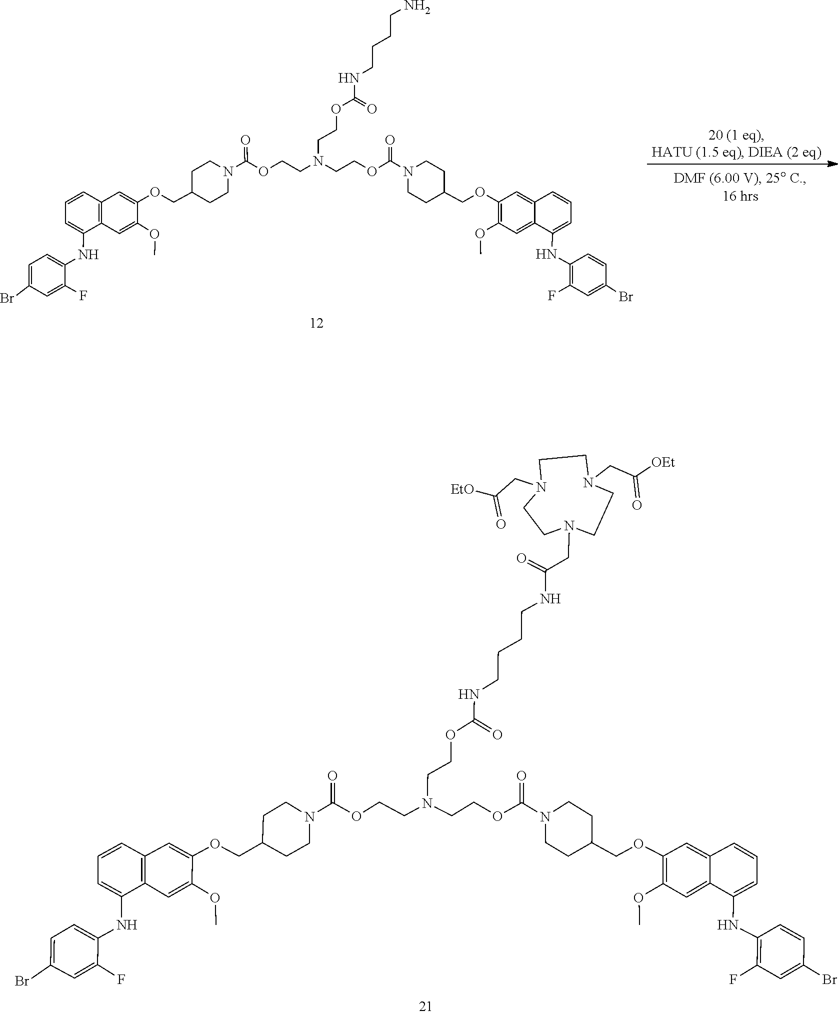 [SMILES: COc1cc2c(Nc3ccc(Br)cc3F)cccc2cc1OCC1CCN(C(=O)OCCN(CCOC(=O)NCCCCN)CCOC(=O)N2CCC(COc3cc4cccc(Nc5ccc(Br)cc5F)c4cc3OC)CC2)CC1 ; CCOC(=O)CN1CCN(CC(=O)NCCCCNC(=O)OCCN(CCOC(=O)N2CCC(COc3cc4cccc(Nc5ccc(Br)cc5F)c4cc3OC)CC2)CCOC(=O)N2CCC(COc3cc4cccc(Nc5ccc(Br)cc5F)c4cc3OC)CC2)CCN(CC(=O)OCC)CC1]

Compound 21 obtained as a yellow oil after work up (pour into water, separate the organic layer, wash the organic layer twice with saturated citric acid, and concentrate under vacuum).

| Entry | 12 (g) | Yield 21 (g) |
|---|---|---|
| 1 | 200 mg | 150 mg |
| 2 | 8 | 8.2 |
| 3 | 88.5 | 80.8 |

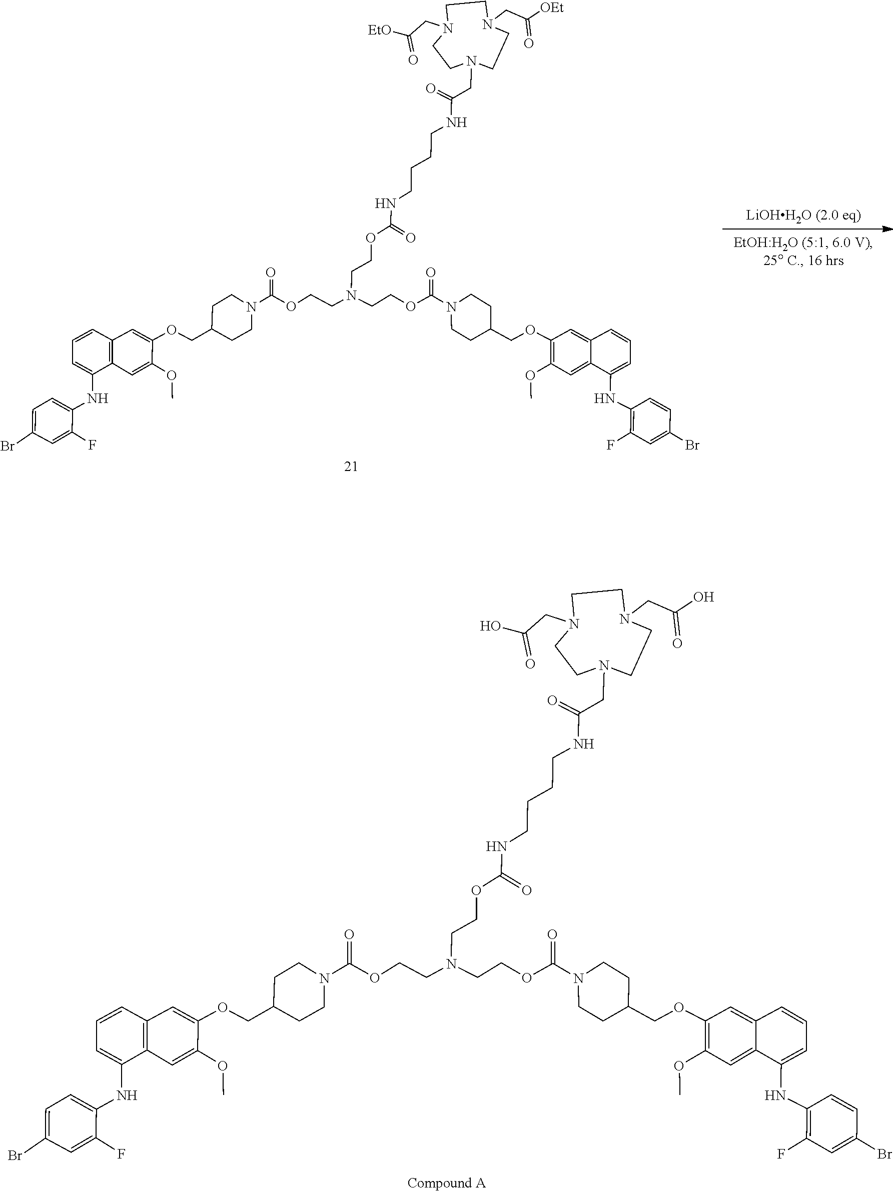 [SMILES: CCOC(=O)CN1CCN(CC(=O)NCCCCNC(=O)OCCN(CCOC(=O)N2CCC(COc3cc4cccc(Nc5ccc(Br)cc5F)c4cc3OC)CC2)CCOC(=O)N2CCC(COc3cc4cccc(Nc5ccc(Br)cc5F)c4cc3OC)CC2)CCN(CC(=O)OCC)CC1 ; COc1cc2c(Nc3ccc(Br)cc3F)cccc2cc1OCC1CCN(C(=O)OCCN(CCOC(=O)NCCCCNC(=O)CN2CCN(CC(=O)O)CCN(CC(=O)O)CC2)CCOC(=O)N2CCC(COc3cc4cccc(Nc5ccc(Br)cc5F)c4cc3OC)CC2)CC1]

From a combination of two runs of the above reaction (8.35 g and 80.8 g of Compound 21), 33.0 g of impure Compound A was obtained. 14.0 g was obtained (99.1% purity) after performing a second prep-HPLC.

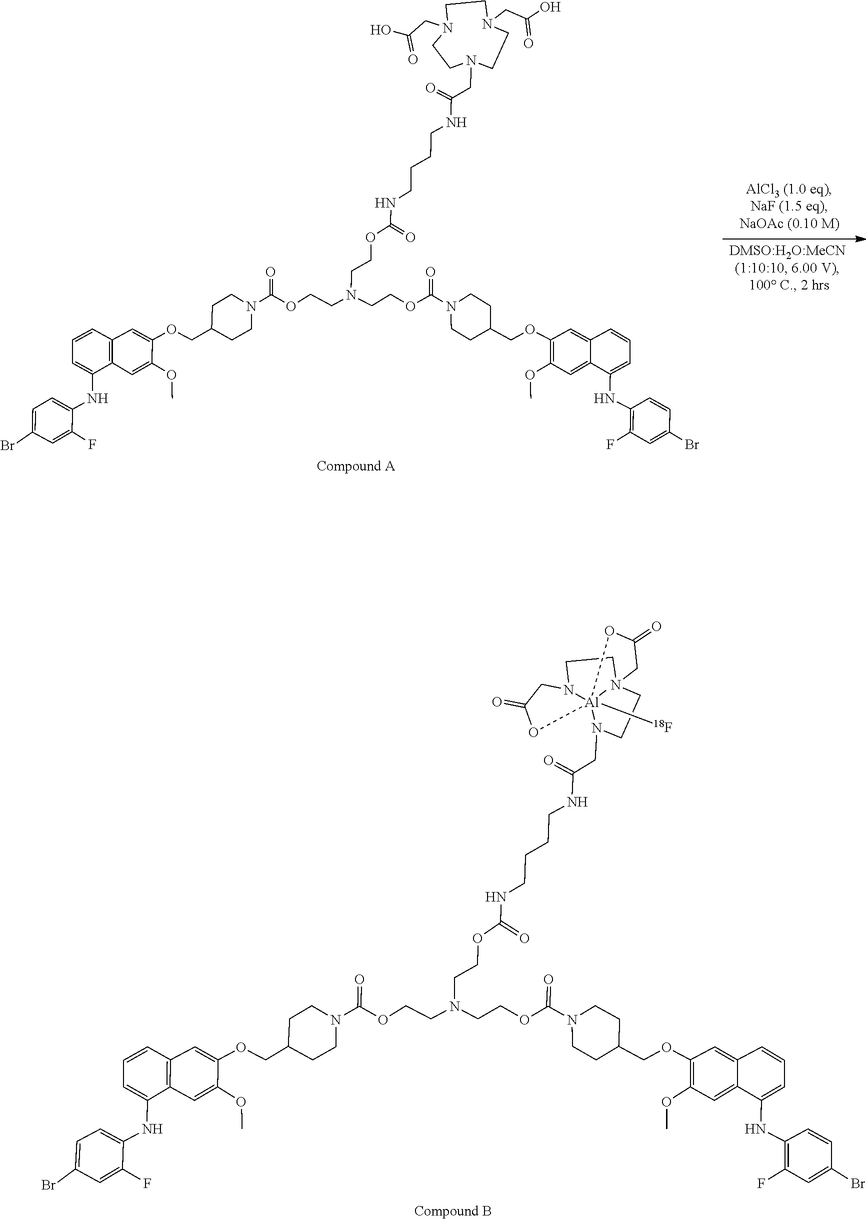

When prepared from 1.10 g of Compound A, 580 mg of Compound B (99.2% HPLC purity) was obtained after purification (prep-HPLC and free-drying).

When prepared from 18.0 g of Compound A, 11.5 g of Compound B (98.6% purity) was obtained.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method of detecting a disease associated with pulmonary vascular remodeling in a subject, the method comprising:
(a) administering to the subject a compound of Formula I or Formula II 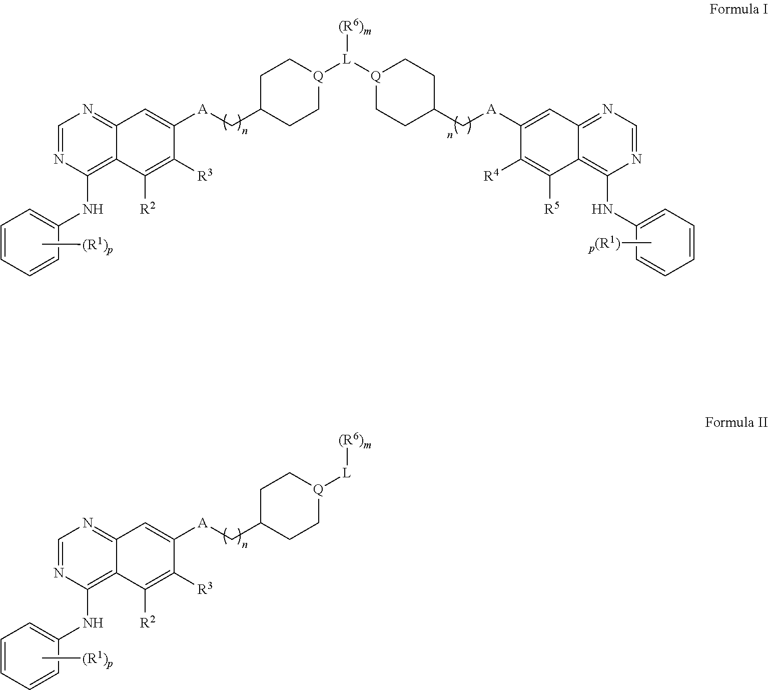
or a pharmaceutically acceptable salt thereof; and
(b) imaging the compound in lungs of the subject using positron emission tomography (PET) to determine uptake of the compound in a pulmonary vasculature of the lungs;
wherein retention of the compound in the pulmonary vasculature reflects vascular remodeling;
and wherein:
R¹ is independently selected at each occurrence from hydroxyl, halogen, C₁₋₃ alkyl, C₁₋₃ alkoxyl, C₁₋₃ alkanoyloxyl, trifluoromethyl, cyano, amino, and nitro;
R², R³, R⁴, and R⁵ are independently selected from hydrogen, hydroxyl, halogen, nitro, trifluoromethyl, cyano, C₁₋₃ alkyl, C₁₋₃ alkoxyl, C₁₋₃ alkylthio, and —NR⁷R⁸,
R⁷ and R⁸ are independently at each occurrence from hydrogen and C₁₋₃ alkyl;
A is —O—, —CH₂—, —S—, —SO—, —SO₂—, —NR⁷CO—, —CONR⁷—, —SO₂NR⁷—, —NR⁷SO₂—, or —NR⁷—;
Q is N or CH;
L is a linker;
R⁶ contains a positron emission tomography (PET) detectable moiety;
n is an integer from 1 to 5;
m is an integer from 1 to 5; and
p is an integer from 1 to 4.

2. The method of claim 1, wherein the PET detectable moiety is an ¹⁸F labeled moiety or ⁶⁸Ga labeled moiety.

3. The method of claim 1, wherein the PET detectable moiety is selected from: 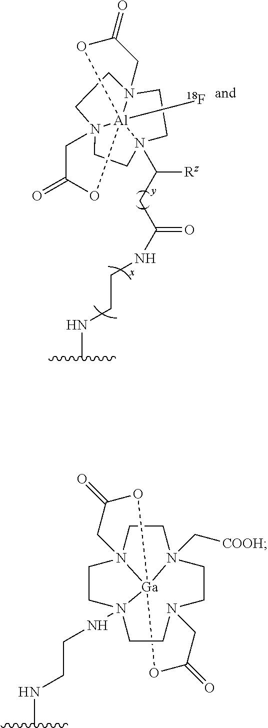
wherein:
x is 1 or 2;
y is 0 or 1; and
R^{Z} is H or COOH.

4. The method of claim 1, wherein the compound is of Formula III 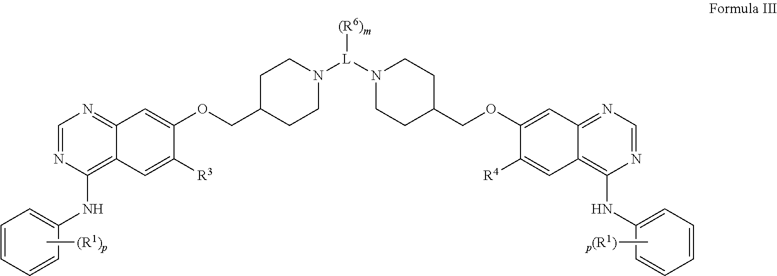
wherein:
R¹ is hydroxy, halogen, C₁₋₃ alkyl, C₁₋₃ alkoxyl, C₁₋₃ alkanoyloxyl, or trifluoromethyl; and
R³ and R⁴ are independently hydrogen, hydroxyl, halogen, C₁₋₃ alkyl, C₁₋₃ alkoxyl, or C₁₋₃ alkylthio.

5. The method of claim 1, wherein the linker is —(CO—R¹⁴)₃N, —(R¹⁴)₃N, —(SO₂R¹⁴)₃N, —(SOR¹⁴)₃N, —(OR¹⁴)₃N, —(O—CO—R¹⁴)₃N, —(CO—O—R¹⁴)₃N, —(CO—R¹⁴)₃CH, —(R¹⁴)₃CH, —(SO₂R¹⁴)₃CH, —(SOR¹⁴)₃CH, —(O—CO—R¹⁴)₃CH, or —(OR¹⁴)₃CH.

6. The method of claim 5, wherein the linker is —(CO—O—R¹⁴)₃N or —(CO—O—R¹⁴)₃CH, wherein R¹⁴ is a C₂₋₄ alkyl.

7. The method of claim 5, wherein the linker is

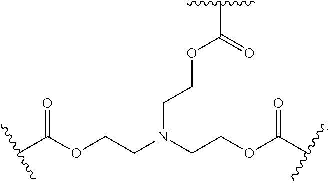 [SMILES: *C(=O)OCCN(CCOC(*)=O)CCOC(*)=O]

8. The method of claim 1, wherein n is 1.

9. The method of claim 1, wherein the compound is of Formula IV:

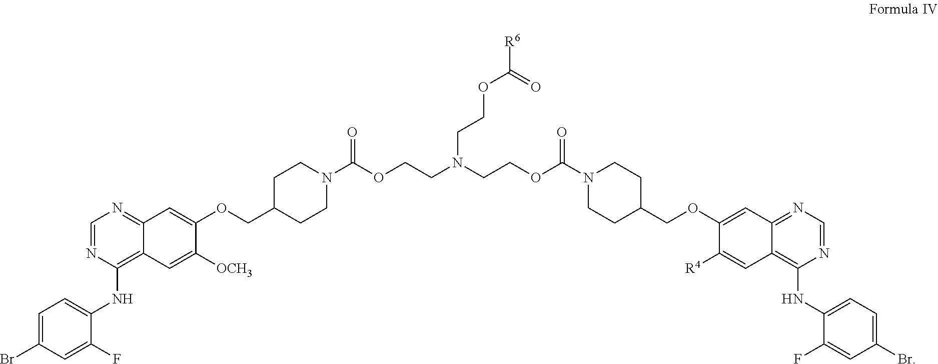

10. The method of claim 1, wherein the compound is selected from: 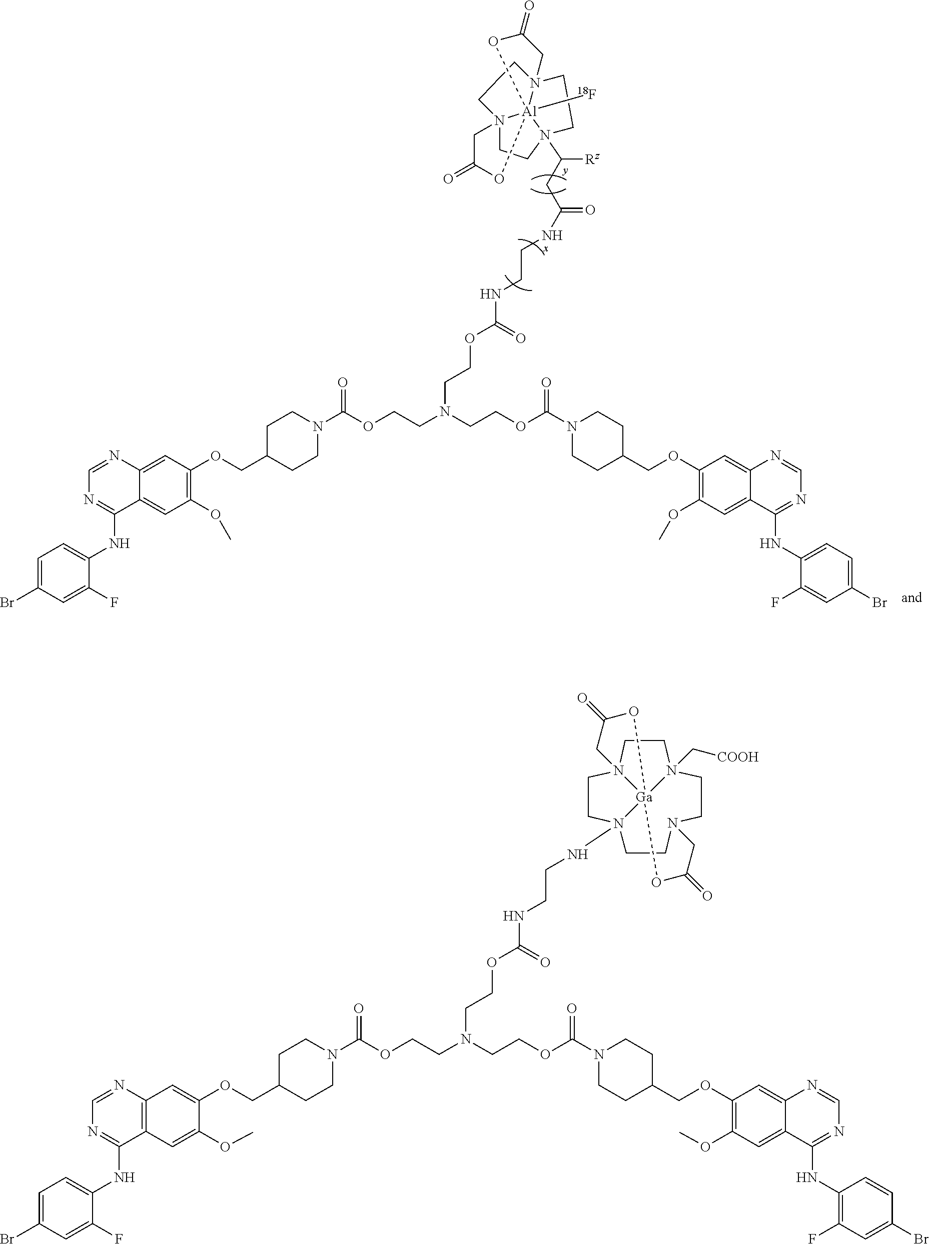
or a pharmaceutically acceptable salt thereof;
wherein:
x is 1 or 2;
y is 0 or 1; and
R^{Z} is H or COOH.

11. The method of claim 1, wherein the disease associated with pulmonary vascular remodeling is pulmonary hypertension.

12. The method of claim 11, wherein the pulmonary hypertension is selected from: pulmonary arterial hypertension (WHO Group I); pulmonary veno-occlusive disease or pulmonary capillary hemangiomatosis (WHO Group I′); persistent pulmonary hypertension of the newborn (WHO Group I″); pulmonary hypertension secondary to left heart disease (WHO Group II); pulmonary hypertension due to lung disease or chronic hypoxia (WHO Group III); chronic arterial obstruction (WHO Group IV); and pulmonary hypertension with unclear or multifactorial mechanisms (WHO Group V).

13. The method of claim 12, wherein the pulmonary hypertension is pulmonary arterial hypertension (PAH).

14. The method of claim 1, wherein the subject is a human.

15. A method of treating a disease associated with pulmonary vascular remodeling in a subject, the method comprising:
(a) administering to the subject a compound of Formula I or Formula II 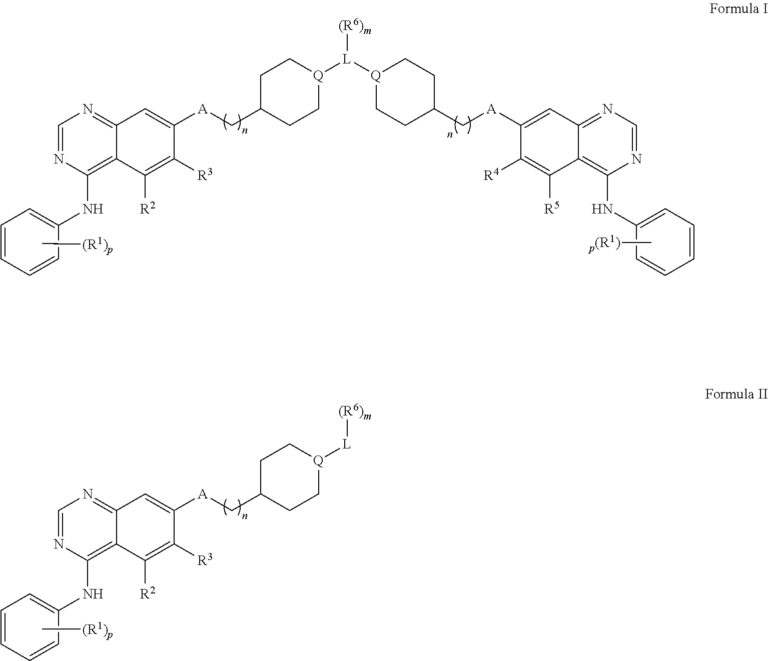
or a pharmaceutically acceptable salt thereof; and
(b) imaging the compound in lungs of the subject using positron emission tomography (PET) to detect uptake of the compound in a pulmonary vasculature of the lungs;
if retention of the compound is detected in the pulmonary vasculature in (b), then (c) administering to the subject a therapy for the disease associated with pulmonary vascular remodeling;
wherein:
R¹ is independently selected at each occurrence from hydroxyl, halogen, C₁₋₃ alkyl, C₁₋₃ alkoxyl, C₁₋₃ alkanoyloxyl, trifluoromethyl, cyano, amino, and nitro;
R², R³, R⁴, and R⁵ are independently selected from hydrogen, hydroxyl, halogen, nitro, trifluoromethyl, cyano, C₁₋₃ alkyl, C₁₋₃ alkoxyl, C₁₋₃ alkylthio, and —NR⁷R⁸,
R⁷ and R⁸ are independently at each occurrence from hydrogen and C₁₋₃ alkyl;
A is —O—, —CH₂—, —S—, —SO—, —SO₂—, —NR⁷CO—, —CONR⁷—, —SO₂NR⁷—, —NR⁷SO₂—, or —NR⁷—;
Q is N or CH;
L is a linker;
R⁶ contains a positron emission tomography (PET) detectable moiety;
n is an integer from 1 to 5;
m is an integer from 1 to 5; and
p is an integer from 1 to 4.

16-28. (canceled)

29. The method of claim 15, wherein the therapy comprises surgery or one or more additional therapeutic agents.

30. (canceled)

31. The method of claim 29, wherein the one or more additional therapeutic agents are selected from a vasodilator, a guanylate cyclase activator, an endothelin receptor antagonist, a phosphodiesterase type 5 inhibitor, a calcium channel blocker, an anticoagulant, digoxin, or a diuretic.

32. A method for imaging a pulmonary vasculature in a subject, the method comprising:
(a) administering to the subject a compound of Formula I or Formula II 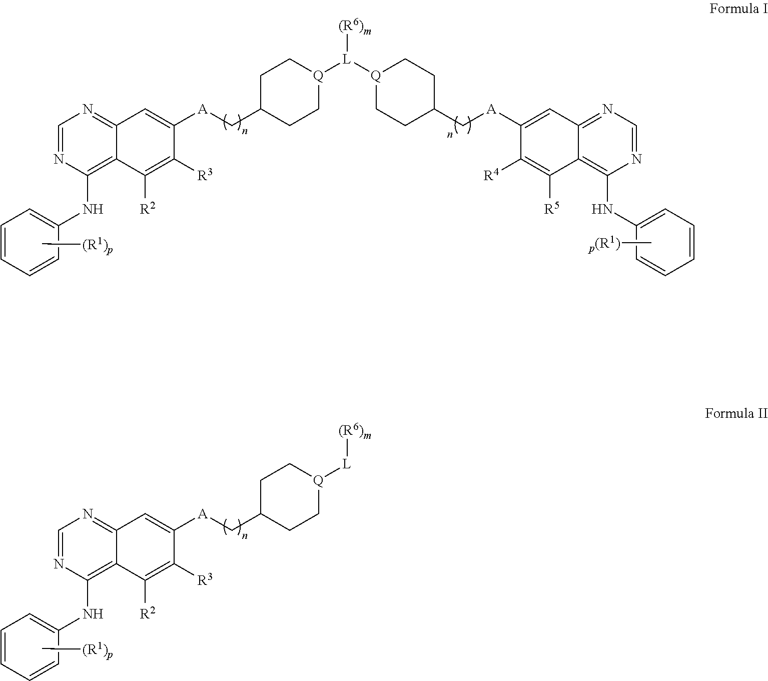
or a pharmaceutically acceptable salt thereof; and
(b) detecting a positron emission tomography (PET) signal in the pulmonary vasculature of the subject;
wherein:
R¹ is independently selected at each occurrence from hydroxyl, halogen, C₁₋₃ alkyl, C₁₋₃ alkoxyl, C₁₋₃ alkanoyloxyl, trifluoromethyl, cyano, amino, and nitro;
R², R³, R⁴, and R⁵ are independently selected from hydrogen, hydroxyl, halogen, nitro, trifluoromethyl, cyano, C₁₋₃ alkyl, C₁₋₃ alkoxyl, C₁₋₃ alkylthio, and —NR⁷R⁸, R⁷ and R⁸ are independently at each occurrence from hydrogen and C₁₋₃ alkyl;
A s —O—, —CH₂—, —S—, —SO—, —SO₂—, —NR⁷CO—, —CONR⁷—, —SO₂NR⁷—, —NR⁷SO₂—, or —NR⁷—;
Q is N or CH;
L is a linker;
R⁶ contains a positron emission tomography (PET) detectable moiety;
n is an integer from 1 to 5;
m is an integer from 1 to 5; and
p is an integer from 1 to 4.

33-42. (canceled)

43. A compound selected from: 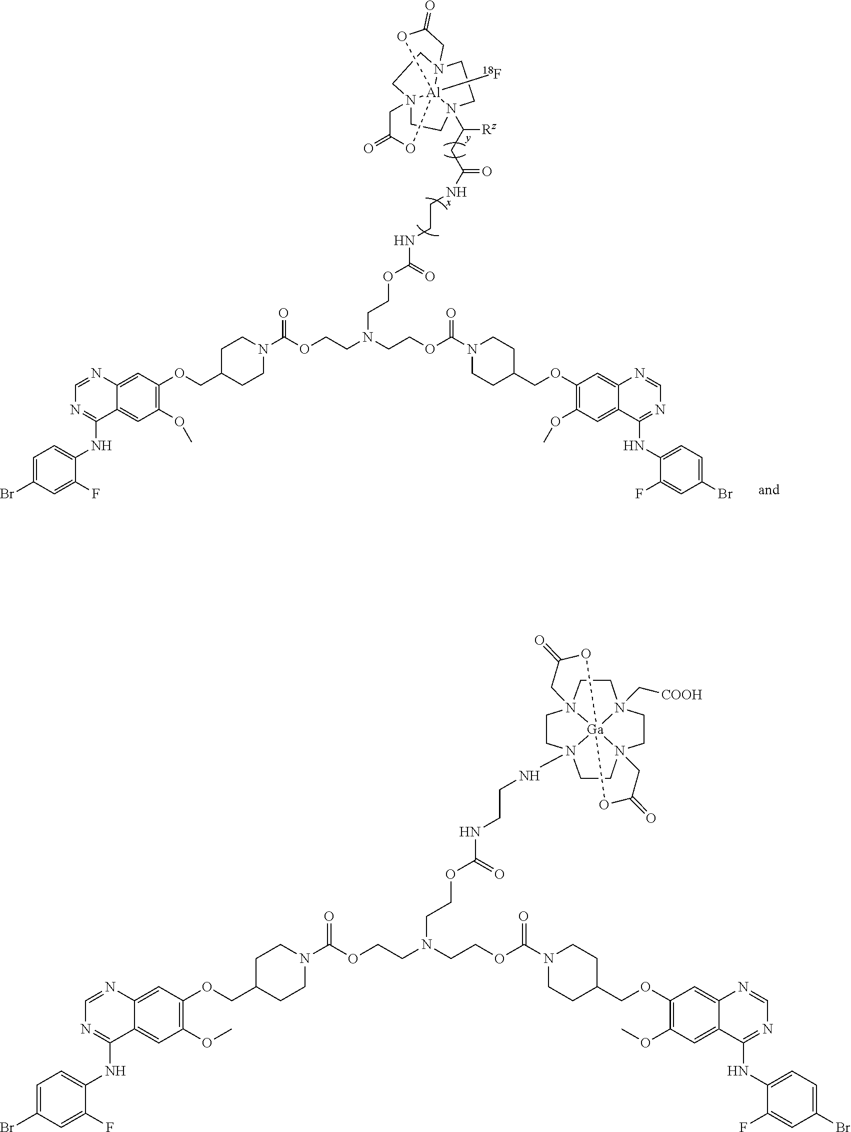
or a pharmaceutically acceptable salt thereof;
wherein:
x is 1 or 2;
y is 0 or 1; and
R^{Z} is H or COOH.

44. A pharmaceutical composition comprising the compound of claim 43, or a pharmaceutically acceptable salt thereof, and a pharmaceutically acceptable carrier or excipient.
